(19) United States
(12) Patent Application Publication
(10) Pub. No.: US 2023/0279474 A1
(43) Pub. Date: Sep. 7, 2023

(54) METHODS FOR SPATIAL ANALYSIS USING BLOCKER OLIGONUCLEOTIDES

(71) Applicant: 10x Genomics, Inc., Pleasanton, CA (US)

(72) Inventor: Layla Katiraee, Castro Valley, CA (US)

(21) Appl. No.: 18/009,093

(22) PCT Filed: Jun. 9, 2021
(86) PCT No.: PCT/US2021/036538

§ 371 Date: Dec. 8, 2022

Related U.S. Application Data
(60) Provisional application No. 63/037,192, filed on Jun. 10, 2020.

(51) Int. Cl. C12Q 1/6832; C12Q 1/6841

(52) U.S. Cl. CPC C12Q 1/6832; C12Q 1/6841

(57) ABSTRACT

Provided herein are methods for blocking undesirable nucleic acids from binding to capture probes using blocker oligonucleotides.

### CROSS-REFERENCE TO RELATED APPLICATIONS

This application claims priority to U.S. Provisional Patent Application No. 63/037,192, filed Jun. 10, 2020. The entire contents of the foregoing applications are incorporated herein by reference.

### BACKGROUND

Cells within a tissue of a subject have differences in cell morphology and/or function due to varied analyte levels (e.g., gene and/or protein expression) within the different cells. The specific position of a cell within a tissue (e.g., the cell's position relative to neighboring cells or the cell's position relative to the tissue microenvironment) can affect, e.g., the cell's morphology, differentiation, fate, viability, proliferation, behavior, and signaling and cross-talk with other cells in the tissue.

Spatial heterogeneity has been previously studied using techniques that only provide data for a small handful of analytes in the context of an intact tissue or a portion of a tissue, or provide a lot of analyte data for single cells, but fail to provide information regarding the position of the single cell in a parent biological sample (e.g., tissue sample).

Non-polyadenylated RNA (e.g., mitochondrial RNA and ribosomal RNA) and other potentially unwanted or undesirable RNA species constitutes a considerable proportion of total nucleic acid pool from the biological sample and can compete with hybridization of target analytes of interest. In certain settings, non-polyadenylated RNA is capable of hybridizing to randomers, poly-adenylated sequences, and even gene specific capture sequences, thus creating increased background signal that interferes with target analyte binding. Further, this non-specific binding represents an expense in the form of lost sequencing data. Removal of undesired species is often done by hybridizing biotinylated probes. However, this subtraction can skew the data by introducing biases and sample loss. It also lengthens the protocol and introduces additional expenses to the user. Because there remains a need to remove such undesirable RNA, this approach simplifies the necessity for multiple enzymatic steps that could affect nucleic acid integrity and function.

### SUMMARY

The present disclosure relates to methods of blocking undesirable nucleic acids (e.g., ribosomal RNAs and/or mitochondrial RNAs) from binding to capture probes, which involves hybridizing the undesirable nucleic acids to blocker oligonucleotides including one or more non-natural nucleic acids (e.g., locked nucleic acids). The non-natural nucleic acids confer the blocker oligonucleotides a high melting temperature, making them unlikely to disassociate throughout the spatial analysis workflow described herein. Because the one or more undesirable nucleic acids do not hybridize to the capture probes, they can be washed away after analytes of interest hybridize to the capture probes. The disclosure is useful for improving more specific and targeted binding between analytes of interest to the capture probes for spatial analysis while introducing minimal biases and sample loss.

In one aspect, provided herein is a method for identifying a location of an analyte of interest in a biological sample comprising: (a) contacting the biological sample with a substrate comprising a plurality of capture probes, wherein a capture probe of the plurality of capture probes comprises (i) a spatial barcode and (ii) a capture domain that specifically hybridizes to the analyte of interest; (b) contacting the biological sample with a plurality of blocker oligonucleotides, wherein a blocker oligonucleotide of the plurality of blocker oligonucleotides is substantially complementary to a sequence of an undesirable nucleic acid molecule in the biological sample; (c) hybridizing the blocker oligonucleotide to the undesirable nucleic acid molecule; (d) extending the capture probe using the analyte of interest as a template to generate an extended capture probe; and (e) amplifying the extended capture probe.

In one aspect, provided herein is a method for increasing the efficiency of capture of an analyte of interest in a biological sample comprising: (a) contacting the biological sample with a substrate comprising a plurality of capture probes, wherein a capture probe of the plurality of capture probes comprises (i) a spatial barcode and (ii) a capture domain that specifically hybridizes to the analyte of interest; (b) contacting the biological sample with a plurality of blocker oligonucleotides, wherein a blocker oligonucleotide of the plurality of blocker oligonucleotides is substantially complementary to a sequence of an undesirable nucleic acid molecule in the biological sample; (c) hybridizing the blocker oligonucleotide to the undesirable nucleic acid molecule; (d) extending the capture probe using the analyte of interest as a template to generate an extended capture probe; and (e) amplifying the extended capture probe. In some embodiments, the efficiency of capture of an analyte of interests increased relative to a reference biological sample to which the plurality of blocker oligonucleotides has not been added.

In some embodiments, provided herein is the method described herein, further comprising determining (i) all or a portion of the sequence of the spatial barcode or a complement thereof, and (ii) all or a portion of the sequence of the analyte of interest from the biological sample, and using the sequences of (i) and (ii) to identify the location of the analyte of interest in the biological sample.

In some embodiments, the plurality of blocker oligonucleotides decreases the concentration of undesirable nucleic acid molecules that hybridize to the plurality of capture probes by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% compared to a reference biological sample to which the plurality of blocker oligonucleotides has not been added.

In some embodiments, the plurality of blocker oligonucleotides increases the concentration of one or more target analytes that hybridize to the plurality of capture domains of capture probes by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, or more compared to a reference biological sample to which the plurality of blocker oligonucleotides has not been added.

In some embodiments, the blocker oligonucleotide comprises at least one non-natural nucleic acid. In some embodiments, the non-natural nucleic acid is a locked nucleic acid.

In some embodiments, the blocker oligonucleotide comprises one or more modifications to its structure. In some embodiments, the one or more modifications is a carbon moiety attached to the locked nucleic acid. In some embodiments, the one or more modifications is a 3′-3′-inverted thymine.

In some embodiments, the blocker oligonucleotide comprises one or more degenerate nucleotides. In some embodiments, the one or more degenerate nucleotides comprise the non-natural nucleic acid.

In some embodiments, the blocker oligonucleotide comprises a sequence that is complementary to a conserved sequence in the undesirable nucleic acid molecule. In some embodiments, the conserved sequence is substantially similar in species selected from one or more of humans, dogs, cats, cows, chickens, rabbits, mice, rats, sheep, horses, amphibians, and reptiles.

In some embodiments, at least 2, at least 3, at least 4, at least 5, at least 6, at least 7, at least 8, at least 9, at least 10, or more blocker oligonucleotides in the plurality of blocker oligonucleotides hybridize to different sequences of the same undesirable nucleic acid molecule.

In some embodiments, the blocker oligonucleotide comprises at least 1, 2, 3, 4, 5, 6, 7, 8, 9, or 10 non-natural nucleic acids. In some embodiments, the blocker oligonucleotide comprises at least 1%, 2%, 3%, 4%, 5%, 10%, 20%, 30%, 40% or 50% non-natural nucleic acids.

In some embodiments, the plurality of blocker oligonucleotides comprises blocker oligonucleotides that target at least 1, at least 10, at least 20, at least 30, at least 40, at least 50, at least 100, at least 200, at least 300, at least 400, at least 500, at least 1000 or more undesired nucleic acids.

In some embodiments, the blocker oligonucleotide is about 25, about 30, about 35, about 40, about 45, or about 50 nucleotides in length.

In some embodiments, the blocker oligonucleotide has a primer melting temperature (Tm) of about 55° C., about 60° C., about 65° C., or about 70° C. In some embodiments, the blocker oligonucleotide comprises at least one LNA, wherein the Tm of the blocker oligonucleotide is at about 10%, about 20%, about 30%, about 40%, about 50%, about 60%, about 70%, about 80%, about 90%, about 100%, or higher than Tm of a reference oligonucleotide without the LNA.

In some embodiments, the blocker oligonucleotide is optically labelled.

In some embodiments, the blocker oligonucleotide is substantially complementary to all or a portion of the sequence of the undesirable nucleic acid molecule in the biological sample.

In some embodiments, at least one blocker oligonucleotide specifically hybridizes to substantially one or more portions of the sequence of the undesirable nucleic acid molecule.

In some embodiments, at least one blocker oligonucleotide specifically hybridizes to substantially the full-length sequence of the undesirable nucleic acid molecule.

In some embodiments, the undesirable nucleic acid molecule an RNA molecule. In some embodiments, the RNA molecule is a transfer RNA (tRNA), a ribosomal RNA (rRNA), a mitochondrial RNA (mtRNA), nuclear RNA, chloroplast RNA, or cytoplasmic RNA, or any combinations thereof.

In some embodiments, the undesirable nucleic acid molecule is an mRNA that encodes hemoglobin, ribulose-1,5-bisphosphate carboxylase/oxygenase (RuBisCo), or glyceraldehyde-3-phosphate dehydrogenase (GAPDH).

In some embodiments, provided herein is the method described herein, further comprising removing a complex of the plurality of blocker oligonucleotide and undesirable nucleic acid molecules. In some embodiments, the removing comprises washing the biological sample.

In some embodiments, the biological sample is permeabilized prior to contacting the biological sample with the plurality of blocker oligonucleotides. In some embodiments, the biological sample is permeabilized concurrently with contacting the biological sample with the plurality of blocker oligonucleotides.

In some embodiments, the biological sample is permeabilized with a permeabilization agent selected from an organic solvent selected from acetone, ethanol, or methanol, a cross-linking agent selected from paraformaldehyde, a detergent selected from saponin, Triton X-100™, Tween-20™, sarkosyl or sodium dodecyl sulfate (SDS), an enzyme selected from trypsin, proteases, including proteinase K, or a combination thereof.

In some embodiments, the biological sample is permeabilized with sarkosyl and/or proteinase K.

In some embodiments, detection of the analyte of interest is associated with a disease or condition.

In some embodiments, the biological sample is a tissue sample.

In some embodiments, the tissue sample is a formalin-fixed, paraffin-embedded (FFPE) tissue sample, a fresh or a frozen tissue sample. In some embodiments, the tissue sample is fixed with ethanol, methanol, acetone, formaldehyde, 2% formaldehyde, paraformaldehyde (PFA), paraformaldehyde-Triton, glutaraldehyde, or combinations thereof.

In some embodiments, the substrate is a slide.

In some embodiments, the biological sample is stained using a detectable label. In some embodiments, the detectable label is H&E.

In some embodiments, the biological sample is imaged. In some embodiments, the biological sample is imaged using brightfield imaging.

In some embodiments, the analyte of interest is an RNA molecule. In some embodiments, the RNA molecule is an mRNA molecule.

In some embodiments, the capture probe further comprises a functional sequence. In some embodiments, the functional sequence is a primer sequence or a complement thereof. In some embodiments, the capture probe further comprises a unique molecular identifying (UMI) sequence or a complement thereof. In some embodiments, the capture probe further comprises an additional primer binding sequence or a complement thereof. In some embodiments, the capture domain comprises a sequence that is substantially complementary to the sequence of the analyte of interest. In some embodiments, the capture domain comprises a sequence that is partially complementary to the sequence of the analyte of interest. In some embodiments, the capture domain comprises a poly-thymine sequence.

In some embodiments, in the extending step, the capture probe is extended at the 3′ end.

In some embodiments, the amplifying is not isothermal. In some embodiments, the amplifying is isothermal.

In some embodiments, a nucleic acid produced from the amplifying step is released from the extended capture probe.

In some embodiments, the determining step comprises sequencing (i) all or a portion of the sequence of the spatial barcode or the complement thereof, and (ii) all or a portion of the sequence of the analyte of interest. In some embodiments, the sequencing is high throughput sequencing. In some embodiments, the sequencing comprises ligating one or more adapters to the nucleic acid.

In one aspect, provided herein is a kit comprising: (a) an array comprising a plurality of capture probes; (b) a plurality of blocker oligonucleotides, wherein a blocker oligonucleotide of the plurality of blocker oligonucleotides is substantially complementary to a sequence of an undesirable nucleic acid molecule in a biological sample, wherein the blocker oligonucleotide comprises at least one non-natural nucleic acid; (c) one or more permeabilization agents; and (d) instructions for using the kit.

In some embodiments, the non-natural nucleic acid is an LNA. In some embodiments, the one or more permeabilization agents sarkosyl and/or proteinase K.

In one aspect, provided herein is a system for decreasing the capture of an undesirable nucleic acid molecule in a biological sample comprising: (a) a substrate comprising a plurality of capture probes, wherein a capture probe of the plurality of capture probes comprises (i) a spatial barcode and (ii) a capture domain that specifically hybridizes to an analyte of interest; and (b) a plurality of blocker oligonucleotides, wherein a blocker oligonucleotide of the plurality of blocker oligonucleotides is substantially complementary to a sequence of the undesirable nucleic acid molecule in the biological sample, wherein the blocker oligonucleotide comprises at least one non-natural nucleic acid.

In some embodiments, provided herein is the system described herein, further comprising a permeabilized biological sample.

In some embodiments, the undesirable nucleic acid molecule is an RNA molecule selected from a transfer RNA (tRNA), a ribosomal RNA (rRNA), a mitochondrial RNA (mtRNA), nuclear RNA, chloroplast RNA, or cytoplasmic RNA, or any combinations thereof.

In some embodiments, the non-natural nucleic acid is a locked nucleic acid.

In some embodiments, the blocker oligonucleotide comprises one or more modifications to its structure. In some embodiments, the one or more modifications is a carbon moiety attached to the locked nucleic acid. In some embodiments, the one or more modifications is a 3′-3′-inverted thymine.

In some embodiments, the blocker oligonucleotide comprises one or more degenerate nucleotides.

In some embodiments, the capture probe further comprises a functional sequence. In some embodiments, the functional sequence is a primer sequence or a complement thereof. In some embodiments, the capture probe further comprises a unique molecular identifying (UMI) sequence or a complement thereof. In some embodiments, the capture probe further comprises an additional primer binding sequence or a complement thereof. In some embodiments, the capture domain comprises a sequence that is substantially complementary to the sequence of the analyte of interest. In some embodiments, the capture domain comprises a sequence that is partially complementary to the sequence of the analyte of interest. In some embodiments, the capture domain comprises a poly-thymine sequence.

All publications, patents, patent applications, and information available on the internet and mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated.

The term “each,” when used in reference to a collection of items, is intended to identify an individual item in the collection but does not necessarily refer to every item in the collection, unless expressly stated otherwise, or unless the context of the usage clearly indicates otherwise.

Various embodiments of the features of this disclosure are described herein. However, it should be understood that such embodiments are provided merely by way of example, and numerous variations, changes, and substitutions can occur to those skilled in the art without departing from the scope of this disclosure. It should also be understood that various alternatives to the specific embodiments described herein are also within the scope of this disclosure.

### BRIEF DESCRIPTION OF THE DRAWINGS

The following drawings illustrate certain embodiments of the features and advantages of this disclosure. These embodiments are not intended to limit the scope of the appended claims in any manner. Like reference symbols in the drawings indicate like elements.

FIG. 1 is a schematic diagram showing an example of a barcoded capture probe, as described herein.

FIG. 2 is a schematic illustrating a cleavable capture probe, wherein the cleaved capture probe can enter into a non-permeabilized cell and bind to target analytes within the sample.

FIG. 3 is a schematic diagram of an exemplary multiplexed spatially-barcoded feature.

FIG. 4 is a schematic diagram of an exemplary analyte capture agent.

FIG. 5 is a schematic diagram depicting an exemplary interaction between a feature-immobilized capture probe 524 and an analyte capture agent 526.

FIGS. 6A, 6B, and 6C are schematics illustrating how streptavidin cell tags can be utilized in an array-based system to produce spatially-barcoded cells or cellular contents.

FIG. 7A shows an exemplary schematic of blocker oligonucleotides that hybridize to an undesirable nucleic acid.

FIG. 7B shows an exemplary embodiment of blocker oligonucleotides hybridizing to an undesirable nucleic acid and an analyte of interest hybridizing to a capture probe.

### DETAILED DESCRIPTION

Disclosed herein are methods, kits, and systems for detecting one or more analytes of interest (e.g., target analyte(s), analyte(s), and the like) in a biological sample. In particular, disclosed herein are methods that block undesirable nucleic acids (e.g., ribosomal RNAs and/or mitochondrial RNAs) from binding to capture probes (e.g., on a spatial array). In some embodiments, the methods involve hybridizing undesirable nucleic acids (e.g., any of the undesirable nucleic acids described herein) with blocker oligonucleotides (e.g., any of the blocker oligonucleotides described herein) including one or more non-natural nucleic acids (e.g., locked nucleic acids). The non-natural nucleic acids confer the blocker oligonucleotides with a high melting temperature, making them unlikely to disassociate throughout the spatial analysis workflow described herein (e.g., a reverse transcription step). Because the one or more undesirable nucleic acids do not hybridize to the capture probes, they can be washed away after analytes of interest hybridize to the capture probes. As a result, less undesirable nucleic acids are extended and/or amplified subsequently following the spatial analysis workflow. Thus, provided herein are methods, kits, and systems for increasing efficiency of capture of targeted analytes from a pool of analytes and the detection of the analytes of interest on a spatial array.

Spatial analysis methodologies and compositions described herein can provide a vast amount of analyte and/or expression data for a variety of analytes within a biological sample at high spatial resolution, while retaining native spatial context. Spatial analysis methods and compositions can include, e.g., the use of a capture probe including a spatial barcode (e.g., a nucleic acid sequence that provides information as to the location or position of an analyte within a cell or a tissue sample (e.g., mammalian cell or a mammalian tissue sample) and a capture domain that is capable of binding to an analyte (e.g., a protein and/or a nucleic acid) produced by and/or present in a cell. Spatial analysis methods and compositions can also include the use of a capture probe having a capture domain that captures an intermediate agent for indirect detection of an analyte. For example, the intermediate agent can include a nucleic acid sequence (e.g., a barcode) associated with the intermediate agent. Detection of the intermediate agent is therefore indicative of the analyte in the cell or tissue sample.

Non-limiting aspects of spatial analysis methodologies and compositions are described in U.S. Pat. Nos. 10,774,374, 10,724,078, 10,480,022, 10,059,990, 10,041,949, 10,002,316, 9,879,313, 9,783,841, 9,727,810, 9,593,365, 8,951,726, 8,604,182, 7,709,198, U.S. Patent Application Publication Nos. 2020/239946, 2020/080136, 2020/0277663, 2020/024641, 2019/330617, 2019/264268, 2020/256867, 2020/224244, 2019/194709, 2019/161796, 2019/085383, 2019/055594, 2018/216161, 2018/051322, 2018/0245142, 2017/241911, 2017/089811, 2017/067096, 2017/029875, 2017/0016053, 2016/108458, 2015/000854, 2013/171621, WO 2018/091676, WO 2020/176788, Rodrigues et al., Science 363(6434):1463-1467, 2019; Lee et al., Nat. Protoc. 10(3):442-458, 2015; Trejo et al., PLOS ONE 14(2):e0212031, 2019; Chen et al., Science 348(6233):aaa6090, 2015; Gao et al., BMC Biol. 15:50, 2017; and Gupta et al., Nature Biotechnol. 36:1197-1202, 2018; the Visium Spatial Gene Expression Reagent Kits User Guide (e.g., Rev D, dated October 2020), and/or the Visium Spatial Tissue Optimization Reagent Kits User Guide (e.g., Rev D, dated October 2020), both of which are available at the 10× Genomics Support Documentation website, and can be used herein in any combination. Further non-limiting aspects of spatial analysis methodologies and compositions are described herein.

Some general terminologies that may be used in this disclosure can be found in Section (I)(b) of WO 2020/176788 and/or U.S. Patent Application Publication No. 2020/0277663. Typically, a “barcode” is a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. For the purpose of this disclosure, an “analyte” can include any biological substance, structure, moiety, or component to be analyzed. The term “target” can similarly refer to an analyte of interest.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I)(c) of WO 2020/176788 and/or U.S. Patent Application Publication No. 2020/0277663. In some embodiments, an analyte can be detected indirectly, such as through detection of an intermediate agent, for example, a connected probe (e.g., a ligation product) or an analyte capture agent (e.g., an oligonucleotide-conjugated antibody), such as those described herein.

A “biological sample” is typically obtained from the subject for analysis using any of a variety of techniques including, but not limited to, biopsy, surgery, and laser capture microscopy (LCM), and generally includes cells and/or other biological material from the subject. In some embodiments, a biological sample can be a tissue section. In some embodiments, a biological sample can be a fixed and/or stained biological sample (e.g., a fixed and/or stained tissue section). Non-limiting examples of stains include histological stains (e.g., hematoxylin and/or eosin) and immunological stains (e.g., fluorescent stains). In some embodiments, a biological sample (e.g., a fixed and/or stained biological sample) can be imaged. Biological samples are also described in Section (I)(d) of WO 2020/176788 and/or U.S. Patent Application Publication No. 2020/0277663.

In some embodiments, a biological sample is permeabilized with one or more permeabilization reagents. For example, permeabilization of a biological sample can facilitate analyte capture. Exemplary permeabilization agents and conditions are described in Section (I)(d)(ii)(13) or the Exemplary Embodiments Section of WO 2020/176788 and/or U.S. Patent Application Publication No. 2020/0277663.

Array-based spatial analysis methods involve the transfer of one or more analytes from a biological sample to an array of features on a substrate, where each feature is associated with a unique spatial location on the array. Subsequent analysis of the transferred analytes includes determining the identity of the analytes and the spatial location of the analytes within the biological sample. The spatial location of an analyte within the biological sample is determined based on the feature to which the analyte is bound (e.g., directly or indirectly) on the array, and the feature's relative spatial location within the array.

A “capture probe” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe includes a barcode (e.g., a spatial barcode and/or a unique molecular identifier (UMI)) and a capture domain). In some embodiments, a capture probe can include a cleavage domain and/or a functional domain (e.g., a primer-binding site, such as for next-generation sequencing (NGS)).

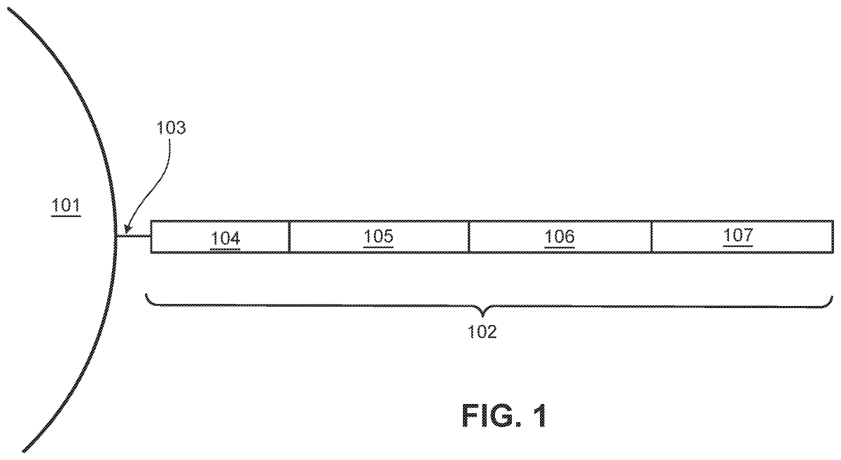

FIG. 1 is a schematic diagram showing an exemplary capture probe, as described herein. As shown, the capture probe 102 is optionally coupled to a feature 101 by a cleavage domain 103, such as a disulfide linker. The capture probe can include a_functional sequence 104 that is useful for subsequent processing. The functional sequence 104 can include all or a part of sequencer specific flow cell attachment sequence (e.g., a P5 or P7 sequence), all or a part of a sequencing primer sequence, (e.g., a R1 primer binding site, a R2 primer binding site), or combinations thereof. The capture probe can also include a spatial barcode 105. The capture probe can also include a unique molecular identifier (UMI) sequence 106. While FIG. 1 shows the spatial barcode 105 as being located upstream (5′) of UMI sequence 106, it is to be understood that capture probes wherein UMI sequence 106 is located upstream (5′) of the spatial barcode 105 is also suitable for use in any of the methods described herein. The capture probe can also include a capture domain 107 to facilitate capture of a target analyte. The capture domain can have a sequence complementary to a sequence of a nucleic acid analyte. The capture domain can have a sequence complementary to a connected probe described herein. The capture domain can have a sequence complementary to a capture handle sequence present in an analyte capture agent. The capture domain can have a sequence complementary to a splint oligonucleotide. Such splint oligonucleotide, in addition to having a sequence complementary to a capture domain of a capture probe, can have a sequence of a nucleic acid analyte, a sequence complementary to a portion of a connected probe described herein, and/or a capture handle sequence described herein.

The functional sequences can generally be selected for compatibility with any of a variety of different sequencing systems, e.g., Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode 105 and functional sequences 104 are common to all of the probes attached to a given feature. In some embodiments, the UMI sequence 106 of a capture probe attached to a given feature is different from the UMI sequence of a different capture probe attached to the given feature.

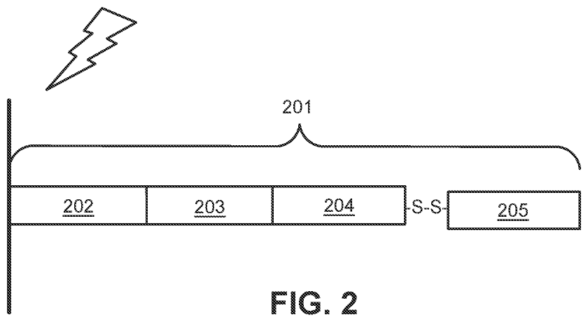

FIG. 2 is a schematic illustrating a cleavable capture probe, wherein the cleaved capture probe can enter into a non-permeabilized cell and bind to analytes within the sample. The capture probe 201 contains a cleavage domain 202, a cell penetrating peptide 203, a reporter molecule 204, and a disulfide bond (—S—S—). 205 represents all other parts of a capture probe, for example a spatial barcode and a capture domain.

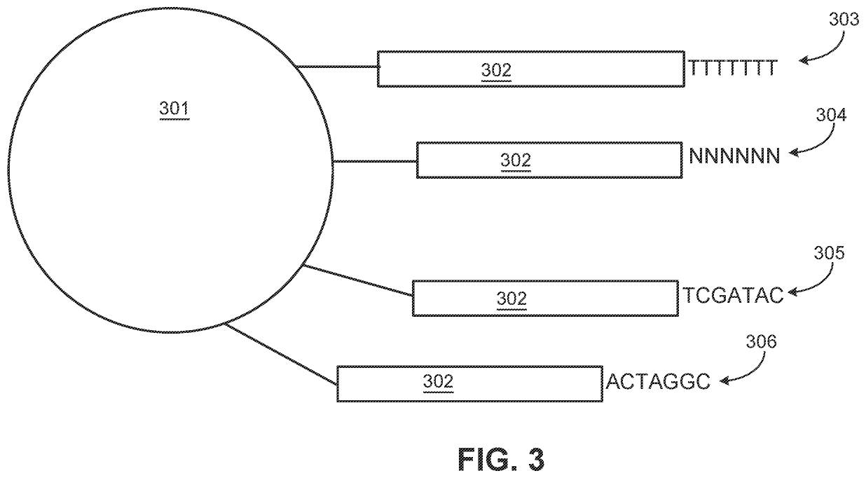

FIG. 3 is a schematic diagram of an exemplary multiplexed spatially-barcoded feature. In FIG. 3, the feature 301 can be coupled to spatially-barcoded capture probes, wherein the spatially-barcoded probes of a particular feature can possess the same spatial barcode, but have different capture domains designed to associate the spatial barcode of the feature with more than one target analyte. For example, a feature may be coupled to four different types of spatially-barcoded capture probes, each type of spatially-barcoded capture probe possessing the spatial barcode 302. One type of capture probe associated with the feature includes the spatial barcode 302 in combination with a poly(T) capture domain 303, designed to capture mRNA target analytes. A second type of capture probe associated with the feature includes the spatial barcode 302 in combination with a random N-mer capture domain 304 for gDNA analysis. A third type of capture probe associated with the feature includes the spatial barcode 302 in combination with a capture domain complementary to a capture handle sequence of an analyte capture agent of interest 305. A fourth type of capture probe associated with the feature includes the spatial barcode 302 in combination with a capture domain that can specifically bind a nucleic acid molecule 306 that can function in a CRISPR assay (e.g., CRISPR/Cas9). While only four different capture probe-barcoded constructs are shown in FIG. 3, capture-probe barcoded constructs can be tailored for analyses of any given analyte associated with a nucleic acid and capable of binding with such a construct. For example, the schemes shown in FIG. 3 can also be used for concurrent analysis of other analytes disclosed herein, including, but not limited to: (a) mRNA, a lineage tracing construct, cell surface or intracellular proteins and metabolites, and gDNA; (b) mRNA, accessible chromatin (e.g., ATAC-seq, DNase-seq, and/or MNase-seq) cell surface or intracellular proteins and metabolites, and a perturbation agent (e.g., a CRISPR crRNA/sgRNA, TALEN, zinc finger nuclease, and/or antisense oligonucleotide as described herein); (c) mRNA, cell surface or intracellular proteins and/or metabolites, a barcoded labelling agent (e.g., the MHC multimers described herein), and a V(D)J sequence of an immune cell receptor (e.g., T-cell receptor). In some embodiments, a perturbation agent can be a small molecule, an antibody, a drug, an aptamer, a miRNA, a physical environmental (e.g., temperature change), or any other known perturbation agents. See, e.g., Section (II)(b) (e.g., subsections (i)-(vi)) of WO 2020/176788 and/or U.S. Patent Application Publication No. 2020/0277663. Generation of capture probes can be achieved by any appropriate method, including those described in Section (II)(d)(ii) of WO 2020/176788 and/or U.S. Patent Application Publication No. 2020/0277663.

In some embodiments, more than one analyte type (e.g., nucleic acids and proteins) from a biological sample can be detected (e.g., simultaneously or sequentially) using any appropriate multiplexing technique, such as those described in Section (IV) of WO 2020/176788 and/or U.S. Patent Application Publication No. 2020/0277663.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) a capture handle sequence. As used herein, the term “analyte binding moiety barcode” refers to a barcode that is associated with or otherwise identifies the analyte binding moiety. As used herein, the term “analyte capture sequence” or “capture handle sequence” refers to a region or moiety configured to hybridize to, bind to, couple to, or otherwise interact with a capture domain of a capture probe. In some embodiments, a capture handle sequence is complementary to a capture domain of a capture probe. In some cases, an analyte binding moiety barcode (or portion thereof) may be able to be removed (e.g., cleaved) from the analyte capture agent.

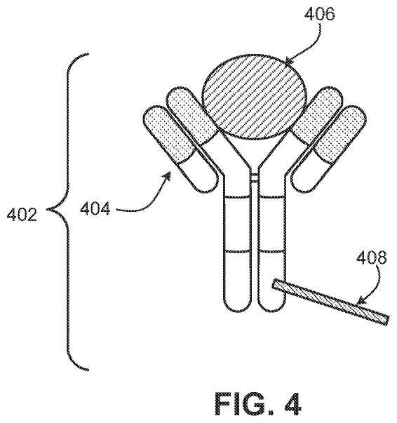

FIG. 4 is a schematic diagram of an exemplary analyte capture agent 402 comprised of an analyte-binding moiety 404 and an analyte-binding moiety barcode domain 408. The exemplary analyte-binding moiety 404 is a molecule capable of binding to an analyte 406 and the analyte capture agent is capable of interacting with a spatially-barcoded capture probe. The analyte-binding moiety can bind to the analyte 406 with high affinity and/or with high specificity. The analyte capture agent can include an analyte-binding moiety barcode domain 408, a nucleotide sequence (e.g., an oligonucleotide), which can hybridize to at least a portion or an entirety of a capture domain of a capture probe. The analyte-binding moiety barcode domain 408 can comprise an analyte binding moiety barcode and a capture handle sequence described herein. The analyte-binding moiety 404 can include a polypeptide and/or an aptamer. The analyte-binding moiety 404 can include an antibody or antibody fragment (e.g., an antigen-binding fragment).

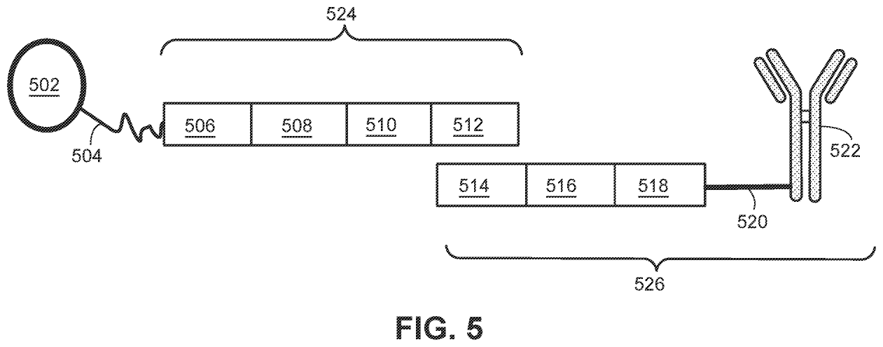

FIG. 5 is a schematic diagram depicting an exemplary interaction between a feature-immobilized capture probe 524 and an analyte capture agent 526. The feature-immobilized capture probe 524 can include a spatial barcode 508 as well as functional sequences 506 and UMI 510, as described elsewhere herein. The capture probe can also include a capture domain 512 that is capable of binding to an analyte capture agent 526. The analyte capture agent 526 can include a functional sequence 518, analyte binding moiety barcode 516, and a capture handle sequence 514 that is capable of binding to the capture domain 512 of the capture probe 524. The analyte capture agent can also include a linker 520 that allows the capture agent barcode domain 516 to couple to the analyte binding moiety 522.

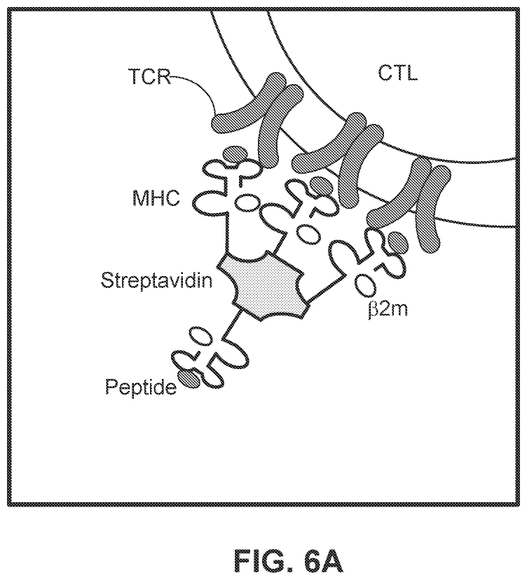

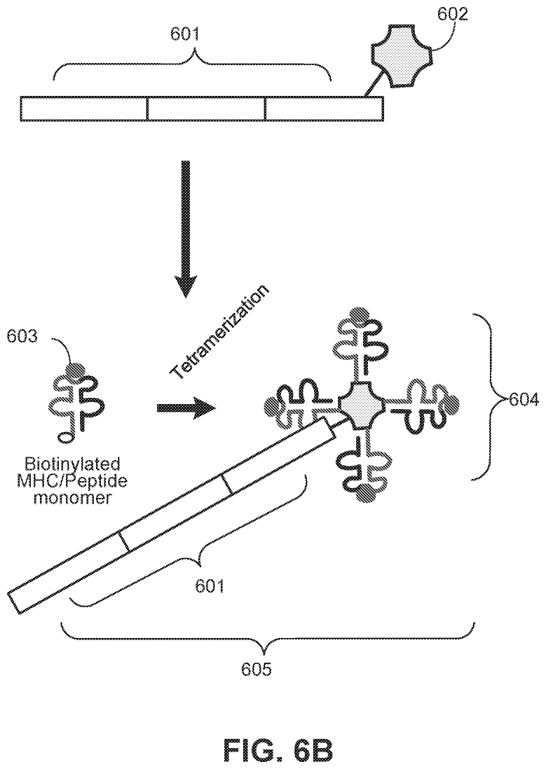

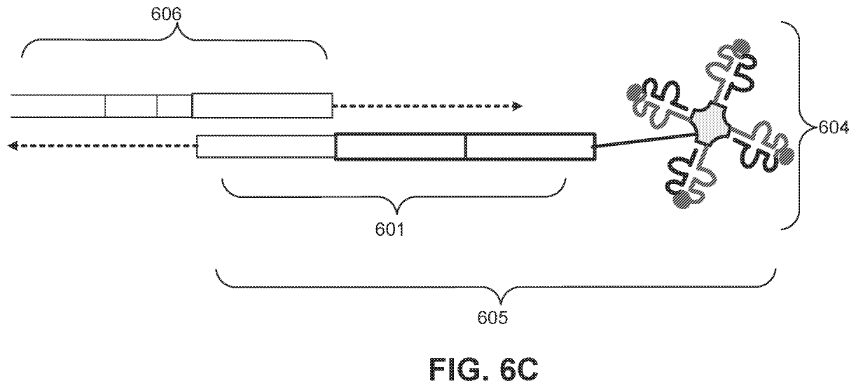

FIGS. 6A, 6B, and 6C are schematics illustrating how streptavidin cell tags can be utilized in an array-based system to produce a spatially-barcoded cell or cellular contents. For example, as shown in FIG. 6A, peptide-bound major histocompatibility complex (MHC) can be individually associated with biotin (β2m) and bound to a streptavidin moiety such that the streptavidin moiety comprises multiple pMHC moieties. Each of these moieties can bind to a TCR such that the streptavidin binds to a target T-cell via multiple MHC/TCR binding interactions. Multiple interactions synergize and can substantially improve binding affinity. Such improved affinity can improve labelling of T-cells and also reduce the likelihood that labels will dissociate from T-cell surfaces. As shown in FIG. 6B, a capture agent barcode domain 601 can be modified with streptavidin 602 and contacted with multiple molecules of biotinylated MHC 603 such that the biotinylated MHC 603 molecules are coupled with the streptavidin conjugated capture agent barcode domain 601. The result is a barcoded MHC multimer complex 605. As shown in FIG. 6B, the capture agent barcode domain sequence 601 can identify the MHC as its associated label and also includes optional functional sequences such as sequences for hybridization with other oligonucleotides. As shown in FIG. 6C, one example oligonucleotide is capture probe 606 that comprises a complementary sequence (e.g., rGrGrG corresponding to C C C), a barcode sequence and other functional sequences, such as, for example, a UMI, an adapter sequence (e.g., comprising a sequencing primer sequence (e.g., R1 or a partial R1 (“pR1”), R2), a flow cell attachment sequence (e.g., P5 or P7 or partial sequences thereof)), etc. In some cases, capture probe 606 may at first be associated with a feature (e.g., a gel bead) and released from the feature. In other embodiments, capture probe 606 can hybridize with a capture agent barcode domain 601 of the MHC-oligonucleotide complex 605. The hybridized oligonucleotides (Spacer C C C and Spacer rGrGrG) can then be extended in primer extension reactions such that constructs comprising sequences that correspond to each of the two spatial barcode sequences (the spatial barcode associated with the capture probe, and the barcode associated with the MHC-oligonucleotide complex) are generated. In some cases, one or both of the corresponding sequences may be a complement of the original sequence in capture probe 606 or capture agent barcode domain 601. In other embodiments, the capture probe and the capture agent barcode domain are ligated together. The resulting constructs can be optionally further processed (e.g., to add any additional sequences and/or for clean-up) and subjected to sequencing. As described elsewhere herein, a sequence derived from the capture probe 606 spatial barcode sequence may be used to identify a feature and the sequence derived from spatial barcode sequence on the capture agent barcode domain 601 may be used to identify the particular peptide MHC complex 604 bound on the surface of the cell (e.g., when using MHC-peptide libraries for screening immune cells or immune cell populations).

Additional description of analyte capture agents can be found in Section (II)(b)(ix) of WO 2020/176788 and/or Section (II)(b)(viii) U.S. Patent Application Publication No. 2020/0277663.

There are at least two methods to associate a spatial barcode with one or more neighboring cells, such that the spatial barcode identifies the one or more cells, and/or contents of the one or more cells, as associated with a particular spatial location. One method is to promote analytes or analyte proxies (e.g., intermediate agents) out of a cell and towards a spatially-barcoded array (e.g., including spatially-barcoded capture probes). Another method is to cleave spatially-barcoded capture probes from an array and promote the spatially-barcoded capture probes towards and/or into or onto the biological sample.

In some cases, capture probes may be configured to prime, replicate, and consequently yield optionally barcoded extension products from a template (e.g., a DNA or RNA template, such as an analyte or an intermediate agent (e.g., a connected probe (e.g., a ligation product) or an analyte capture agent), or a portion thereof), or derivatives thereof (see, e.g., Section (II)(b)(vii) of WO 2020/176788 and/or U.S. Patent Application Publication No. 2020/0277663 regarding extended capture probes). In some cases, capture probes may be configured to form a connected probe (e.g., a ligation product) with a template (e.g., a DNA or RNA template, such as an analyte or an intermediate agent, or portion thereof), thereby creating ligations products that serve as proxies for a template.

As used herein, an “extended capture probe” refers to a capture probe having additional nucleotides added to the terminus (e.g., 3′ or 5′ end) of the capture probe thereby extending the overall length of the capture probe. For example, an “extended 3′ end” indicates additional nucleotides were added to the most 3′ nucleotide of the capture probe to extend the length of the capture probe, for example, by polymerization reactions used to extend nucleic acid molecules including templated polymerization catalyzed by a polymerase (e.g., a DNA polymerase or a reverse transcriptase). In some embodiments, extending the capture probe includes adding to a 3′ end of a capture probe a nucleic acid sequence that is complementary to a nucleic acid sequence of an analyte or intermediate agent specifically bound to the capture domain of the capture probe. In some embodiments, the capture probe is extended using reverse transcription. In some embodiments, the capture probe is extended using one or more DNA polymerases. The extended capture probes include the sequence of the capture probe and the sequence of the spatial barcode of the capture probe.

In some embodiments, extended capture probes are amplified (e.g., in bulk solution or on the array) to yield quantities that are sufficient for downstream analysis, e.g., via DNA sequencing. In some embodiments, extended capture probes (e.g., DNA molecules) act as templates for an amplification reaction (e.g., a polymerase chain reaction).

Additional variants of spatial analysis methods, including in some embodiments, an imaging step, are described in Section (II)(a) of WO 2020/176788 and/or U.S. Patent Application Publication No. 2020/0277663. Analysis of captured analytes (and/or intermediate agents or portions thereof), for example, including sample removal, extension of capture probes, sequencing (e.g., of a cleaved extended capture probe and/or a cDNA molecule complementary to an extended capture probe), sequencing on the array (e.g., using, for example, in situ hybridization or in situ ligation approaches), temporal analysis, and/or proximity capture, is described in Section (II)(g) of WO 2020/176788 and/or U.S. Patent Application Publication No. 2020/0277663. Some quality control measures are described in Section (II)(h) of WO 2020/176788 and/or U.S. Patent Application Publication No. 2020/0277663.

Spatial information can provide information of biological and/or medical importance. For example, the methods and compositions described herein can allow for: identification of one or more biomarkers (e.g., diagnostic, prognostic, and/or for determination of efficacy of a treatment) of a disease or disorder; identification of a candidate drug target for treatment of a disease or disorder; identification (e.g., diagnosis) of a subject as having a disease or disorder; identification of stage and/or prognosis of a disease or disorder in a subject; identification of a subject as having an increased likelihood of developing a disease or disorder; monitoring of progression of a disease or disorder in a subject; determination of efficacy of a treatment of a disease or disorder in a subject; identification of a patient subpopulation for which a treatment is effective for a disease or disorder; modification of a treatment of a subject with a disease or disorder; selection of a subject for participation in a clinical trial; and/or selection of a treatment for a subject with a disease or disorder.

Spatial information can provide information of biological importance. For example, the methods and compositions described herein can allow for: identification of transcriptome and/or proteome expression profiles (e.g., in healthy and/or diseased tissue); identification of multiple analyte types in close proximity (e.g., nearest neighbor analysis); determination of up- and/or down-regulated genes and/or proteins in diseased tissue; characterization of tumor microenvironments; characterization of tumor immune responses; characterization of cells types and their co-localization in tissue; and identification of genetic variants within tissues (e.g., based on gene and/or protein expression profiles associated with specific disease or disorder biomarkers).

Typically, for spatial array-based methods, a substrate functions as a support for direct or indirect attachment of capture probes to features of the array. A “feature” is an entity that acts as a support or repository for various molecular entities used in spatial analysis. In some embodiments, some or all of the features in an array are functionalized for analyte capture. Exemplary substrates are described in Section (II)(c) of WO 2020/176788 and/or U.S. Patent Application Publication No. 2020/0277663. Exemplary features and geometric attributes of an array can be found in Sections (II)(d)(i), (II)(d)(iii), and (II)(d)(iv) of WO 2020/176788 and/or U.S. Patent Application Publication No. 2020/0277663.

Generally, analytes and/or intermediate agents (or portions thereof) can be captured when contacting a biological sample with a substrate including capture probes (e.g., a substrate with capture probes embedded, spotted, printed, fabricated on the substrate, or a substrate with features (e.g., beads, wells) comprising capture probes). As used herein, “contact,” “contacted,” and/or “contacting,” a biological sample with a substrate refers to any contact (e.g., direct or indirect) such that capture probes can interact (e.g., bind covalently or non-covalently (e.g., hybridize)) with analytes from the biological sample. Capture can be achieved actively (e.g., using electrophoresis) or passively (e.g., using diffusion). Analyte capture is further described in Section (II)(e) of WO 2020/176788 and/or U.S. Patent Application Publication No. 2020/0277663.

In some cases, spatial analysis can be performed by attaching and/or introducing a molecule (e.g., a peptide, a lipid, or a nucleic acid molecule) having a barcode (e.g., a spatial barcode) to a biological sample (e.g., to a cell in a biological sample). In some embodiments, a plurality of molecules (e.g., a plurality of nucleic acid molecules) having a plurality of barcodes (e.g., a plurality of spatial barcodes) are introduced to a biological sample (e.g., to a plurality of cells in a biological sample) for use in spatial analysis. In some embodiments, after attaching and/or introducing a molecule having a barcode to a biological sample, the biological sample can be physically separated (e.g., dissociated) into single cells or cell groups for analysis. Some such methods of spatial analysis are described in Section (III) of WO 2020/176788 and/or U.S. Patent Application Publication No. 2020/0277663.

In some cases, spatial analysis can be performed by detecting multiple oligonucleotides that hybridize to an analyte. In some instances, for example, spatial analysis can be performed using RNA-templated ligation (RTL). Methods of RTL have been described previously. See, e.g., Credle et al., Nucleic Acids Res. 2017 Aug. 21; 45(14):e128. Typically, RTL includes hybridization of two oligonucleotides to adjacent sequences on an analyte (e.g., an RNA molecule, such as an mRNA molecule). In some instances, the oligonucleotides are DNA molecules. In some instances, one of the oligonucleotides includes at least two ribonucleic acid bases at the 3′ end and/or the other oligonucleotide includes a phosphorylated nucleotide at the 5′ end. In some instances, one of the two oligonucleotides includes a capture domain (e.g., a poly(A) sequence, a non-homopolymeric sequence). After hybridization to the analyte, a ligase (e.g., SplintR ligase) ligates the two oligonucleotides together, creating a connected probe (e.g., a ligation product). In some instances, the two oligonucleotides hybridize to sequences that are not adjacent to one another. For example, hybridization of the two oligonucleotides creates a gap between the hybridized oligonucleotides. In some instances, a polymerase (e.g., a DNA polymerase) can extend one of the oligonucleotides prior to ligation. After ligation, the connected probe (e.g., a ligation product) is released from the analyte. In some instances, the connected probe (e.g., a ligation product) is released using an endonuclease (e.g., RNAse H). The released connected probe (e.g., a ligation product) can then be captured by capture probes (e.g., instead of direct capture of an analyte) on an array, optionally amplified, and sequenced, thus determining the location and optionally the abundance of the analyte in the biological sample.

During analysis of spatial information, sequence information for a spatial barcode associated with an analyte is obtained, and the sequence information can be used to provide information about the spatial distribution of the analyte in the biological sample. Various methods can be used to obtain the spatial information. In some embodiments, specific capture probes and the analytes they capture are associated with specific locations in an array of features on a substrate. For example, specific spatial barcodes can be associated with specific array locations prior to array fabrication, and the sequences of the spatial barcodes can be stored (e.g., in a database) along with specific array location information, so that each spatial barcode uniquely maps to a particular array location.

Alternatively, specific spatial barcodes can be deposited at predetermined locations in an array of features during fabrication such that at each location, only one type of spatial barcode is present so that spatial barcodes are uniquely associated with a single feature of the array. Where necessary, the arrays can be decoded using any of the methods described herein so that spatial barcodes are uniquely associated with array feature locations, and this mapping can be stored as described above.

When sequence information is obtained for capture probes and/or analytes during analysis of spatial information, the locations of the capture probes and/or analytes can be determined by referring to the stored information that uniquely associates each spatial barcode with an array feature location. In this manner, specific capture probes and captured analytes are associated with specific locations in the array of features. Each array feature location represents a position relative to a coordinate reference point (e.g., an array location, a fiducial marker) for the array. Accordingly, each feature location has an “address” or location in the coordinate space of the array.

Some exemplary spatial analysis workflows are described in the Exemplary Embodiments section of WO 2020/176788 and/or U.S. Patent Application Publication No. 2020/0277663. See, for example, the Exemplary embodiment starting with “In some non-limiting examples of the workflows described herein, the sample can be immersed . . . ” of WO 2020/176788 and/or U.S. Patent Application Publication No. 2020/0277663. See also, e.g., the Visium Spatial Gene Expression Reagent Kits User Guide (e.g., Rev D, dated October 2020), and/or the Visium Spatial Tissue Optimization Reagent Kits User Guide (e.g., Rev D, dated October 2020). In some embodiments, spatial analysis can be performed using dedicated hardware and/or software, such as any of the systems described in Sections (II)(e)(ii) and/or (V) of WO 2020/176788 and/or U.S. Patent Application Publication No. 2020/0277663, or any of one or more of the devices or methods described in Sections Control Slide for Imaging, Methods of Using Control Slides and Substrates for, Systems of Using Control Slides and Substrates for Imaging, and/or Sample and Array Alignment Devices and Methods, Informational labels of WO 2020/123320.

Suitable systems for performing spatial analysis can include components such as a chamber (e.g., a flow cell or sealable, fluid-tight chamber) for containing a biological sample. The biological sample can be mounted for example, in a biological sample holder. One or more fluid chambers can be connected to the chamber and/or the sample holder via fluid conduits, and fluids can be delivered into the chamber and/or sample holder via fluidic pumps, vacuum sources, or other devices coupled to the fluid conduits that create a pressure gradient to drive fluid flow. One or more valves can also be connected to fluid conduits to regulate the flow of reagents from reservoirs to the chamber and/or sample holder.

The systems can optionally include a control unit that includes one or more electronic processors, an input interface, an output interface (such as a display), and a storage unit (e.g., a solid state storage medium such as, but not limited to, a magnetic, optical, or other solid state, persistent, writeable and/or re-writeable storage medium). The control unit can optionally be connected to one or more remote devices via a network. The control unit (and components thereof) can generally perform any of the steps and functions described herein. Where the system is connected to a remote device, the remote device (or devices) can perform any of the steps or features described herein. The systems can optionally include one or more detectors (e.g., CCD, CMOS) used to capture images. The systems can also optionally include one or more light sources (e.g., LED-based, diode-based, lasers) for illuminating a sample, a substrate with features, analytes from a biological sample captured on a substrate, and various control and calibration media.

The systems can optionally include software instructions encoded and/or implemented in one or more of tangible storage media and hardware components such as application specific integrated circuits. The software instructions, when executed by a control unit (and in particular, an electronic processor) or an integrated circuit, can cause the control unit, integrated circuit, or other component executing the software instructions to perform any of the method steps or functions described herein.

In some cases, the systems described herein can detect (e.g., register an image) the biological sample on the array. Exemplary methods to detect the biological sample on an array are described in PCT Application No. 2020/061064 and/or U.S. patent application Ser. No. 16/951,854.

Prior to transferring analytes from the biological sample to the array of features on the substrate, the biological sample can be aligned with the array. Alignment of a biological sample and an array of features including capture probes can facilitate spatial analysis, which can be used to detect differences in analyte presence and/or level within different positions in the biological sample, for example, to generate a three-dimensional map of the analyte presence and/or level. Exemplary methods to generate a two- and/or three-dimensional map of the analyte presence and/or level are described in PCT Application No. 2020/053655 and spatial analysis methods are generally described in WO 2020/061108 and/or U.S. patent application Ser. No. 16/951,864.

In some cases, a map of analyte presence and/or level can be aligned to an image of a biological sample using one or more fiducial markers, e.g., objects placed in the field of view of an imaging system which appear in the image produced, as described in the Substrate Attributes Section, Control Slide for Imaging Section of WO 2020/123320, PCT Application No. 2020/061066, and/or U.S. patent application Ser. No. 16/951,843. Fiducial markers can be used as a point of reference or measurement scale for alignment (e.g., to align a sample and an array, to align two substrates, to determine a location of a sample or array on a substrate relative to a fiducial marker) and/or for quantitative measurements of sizes and/or distances.

Methods of Blocking Undesirable Nucleic Acid Binding to Capture Probes using Blocker Oligonucleotides

The present disclosure provides methods for blocking undesirable nucleic acids (e.g., ribosomal RNAs and/or mitochondrial RNAs) from binding to capture probes using blocker oligonucleotides. For example, the blocker oligonucleotides can hybridize to the undesirable nucleic acids (e.g., at conserved regions) such that the undesirable nucleic acids do not hybridize to the capture probes (e.g., on a spatial substrate or array as described herein). In some embodiments, the blocker oligonucleotide-bound duplex regions prevents the undesirable nucleic acid from being extended and/or amplified following the spatial analysis workflow. In some embodiments, hybridization of the blocker oligonucleotides reduces sequencing reads of the undesirable nucleic acids, which in turn increases sequencing reads of one or more target analytes (e.g., target nucleic acids or analytes of interest).

In some embodiments, hybridization of the blocker oligonucleotides can decrease the population (e.g., in terms of concentration) of undesirable nucleic acids that hybridize to capture probes by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% as compared to the population of the same undesirable nucleic acids that hybridize to capture probes from a reference biological sample to which the blocker oligonucleotides are not added. In some embodiments, hybridization of the blocker oligonucleotides can increase the population (e.g., in terms of concentration) of one or more target analytes (e.g., an mRNA target) that hybridize to capture probes by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% as compared to the population of the one or more target analytes that hybridize to capture probes from a reference biological sample to which the blocker oligonucleotides are not added.

In some embodiments, hybridization of the blocker oligonucleotides can decrease the occurrence of extension and/or amplification of the undesirable nucleic acids by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% as compared to the occurrence of similar events from a reference biological sample to which the blocker oligonucleotides are not added.

In some embodiments, hybridization of the blocker oligonucleotides can increase efficiency of identifying a location of an analyte of interest in a biological sample by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, or more as compared to the efficiency of identifying the location of the same analyte of interest in a reference biological sample to which the blocker oligonucleotides are not added. For example, in some embodiments, hybridization of the blocker oligonucleotides can decrease sequencing reads of the undesirable nucleic acids by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% as compared to sequencing reads of the undesirable nucleic acids from a reference biological sample to which the blocker oligonucleotides are not added. Consequently, hybridization of the blocker oligonucleotides can increase sequencing reads of one or more target analytes (e.g., target nucleic acids or analytes of interest) by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% about as compared to sequencing reads of the one or more target analytes from the reference biological sample to which the blocker oligonucleotides are not added.

Methods disclosed herein can be performed on any type of sample as disclosed herein. In some embodiments, the sample is a tissue sample. In some embodiments, the sample is a culture of cells. In some embodiments, the sample is a fresh tissue. In some embodiments, the sample is a frozen sample. In some embodiments, the sample was previously frozen. In some embodiments, the sample is a formalin-fixed, paraffin embedded (FFPE) sample. FFPE samples generally are heavily cross-linked and fragmented, and therefore this type of sample allows for limited RNA recovery using conventional detection techniques. In certain embodiments, methods provided herein are less affected by RNA degradation associated with FFPE fixation than other methods (e.g., methods that take advantage of oligo-dT capture and reverse transcription of mRNA). In certain embodiments, methods provided herein enable sensitive measurement of specific genes of interest that otherwise might be missed with a whole transcriptomic approach. In certain embodiments, methods provided herein enable sensitive measurement of target nucleic acids without wasting sequencing reads on undesirable nucleic acids.

As an alternative to formalin fixation described above, a biological sample can be fixed in any of a variety of other fixatives to preserve the biological structure of the sample prior to analysis. For example, a sample can be fixed via immersion in ethanol, methanol, acetone, formaldehyde (e.g., 2% formaldehyde), paraformaldehyde (PFA), paraformaldehyde-Triton, glutaraldehyde, or combinations thereof. In some embodiments, acetone fixation is used with fresh frozen samples, which can include, but are not limited to, cortex tissue, mouse olfactory bulb, human brain tumor, human post-mortem brain, and breast cancer samples. In some embodiments, a compatible fixation method is chosen and/or optimized based on a desired workflow. For example, formaldehyde fixation may be chosen as compatible for workflows using IHC/IF protocols for protein visualization. As another example, methanol fixation may be chosen for workflows emphasizing RNA/DNA library quality. Acetone fixation may be chosen in some applications to permeabilize the tissue. When acetone fixation is performed, pre-permeabilization steps (described below) may not be performed. Alternatively, acetone fixation can be performed in conjunction with permeabilization steps.

In some embodiments, a biological sample (e.g., a tissue section) can be imaged using any of the imaging techniques described herein. In some embodiments, the biological sample can be fixed with methanol, stained with hematoxylin and eosin (H&E), and imaged. In some instances, the sample is imaged using one or more detectable markers (e.g., a radioisotope, a fluorescent, or a chemiluminescent moiety) designed to associate with one or more analytes. In some embodiments, fixing, staining, and/or imaging occur before one or more probes (e.g., capture probes) are hybridized to the sample. In some embodiments, staining occurs at the same time one or more probes are hybridized to the sample. In some embodiments, staining and imaging occurs after one or more probes are hybridized to the sample. Some embodiments of any of the workflows described herein can further include a destaining step (e.g., a hematoxylin and eosin destaining step). In some embodiments, destaining occurs after imaging of the sample and prior to permeabilizing the sample. For example, destaining can be performed by performing one or more (e.g., one, two, three, four, or five) washing steps (e.g., one or more (e.g., one, two, three, four, or five) washing steps performed using a buffer, including HCl). The images can be used to map spatial gene expression patterns back to the biological sample. A permeabilization enzyme can be used to permeabilize the biological sample directly on the slide.

In some embodiments, the FFPE sample is deparaffinized, permeabilized, equilibrated, and blocked before target probe oligonucleotides are added. In some embodiments, deparaffinization uses xylenes. In some embodiments, deparaffinization includes multiple washes with xylenes. In some embodiments, the deparaffinized sample can be treated to de-crosslink methylene bridges in a single step, e.g., at about 65° C., about 66° C., about 67° C., about 68° C., about 69° C., or about 70° C. In some embodiments, deparaffinization includes multiple washes with xylenes followed by removal of xylenes using multiple rounds of graded alcohol washes followed by washing the sample with water. In some aspects, the water is deionized water. In some embodiments, equilibrating and blocking includes incubating the sample in a pre-Hyb buffer. In some embodiments, the pre-Hyb buffer includes yeast tRNA. In some embodiments, the pre-Hyb buffer includes rRNA. In some instances, the rRNA in the pre-Hyb buffer includes E. coli rRNA. In some embodiments, permeabilizing a sample includes washing the sample with a phosphate buffer. In some embodiments, the buffer is PBS. In some embodiments, the buffer is PBST.

(a) Blocker Oligonucleotides

In some embodiments, the methods described herein include contacting the biological sample with a plurality of blocker oligonucleotides. In some embodiments, the biological sample is contacted with the blocker oligonucleotides before being contacted with the spatial array described herein. In some embodiments, the biological sample is contacted with the blocker oligonucleotides at substantially the same time of being contacted with the spatial array described herein. In some embodiments, the biological sample is contacted with the blocker oligonucleotides after being contacted with the spatial array described herein.

In some embodiments, the methods described herein include permeabilizing the biological sample, e.g., using a permeabilization buffer. In some embodiments, the permeabilization is a partial permeabilization. For example, during staining and/or imaging, less than 10%, 9%, 8%, 7%, 6%, 5%, 4%, 3%, 2%, 1%, or less cells in the biological sample are permeabilized. In some embodiments, the biological sample is contacted with the blocker oligonucleotides before being permeabilized. In some embodiments, the biological sample is contacted with the blocker oligonucleotides at substantially the same time of being permeabilized. In some embodiments, the biological sample is contacted with the blocker oligonucleotides after being permeabilized. In some embodiments, the blocker oligonucleotides are added to a permeabilization buffer (e.g., a permeabilization master mix).

In some embodiments, the methods described herein include hybridizing an analyte of interest in the biological sample to the capture probe described herein. In some embodiments, the biological sample is contacted with the blocker oligonucleotides before the analyte of interest is hybridized to the capture probe. In some embodiments, the biological sample is contacted with the blocker oligonucleotides at substantially the same time as the analyte of interest is hybridized to the capture probe. In some embodiments, the biological sample is contacted with the blocker oligonucleotides after the analyte of interest is hybridized to the capture probe.

In some embodiments, the methods described herein include extending the capture probe using the analyte of interest as a template to generate an extended capture probe. In some embodiments, the extension of the capture probe is achieved by reverse transcription (RT). In some embodiments, the RT is performed in a pre-mixed solution. In some embodiments, the RT is performed using a master mix solution. In some embodiments, the blocker oligonucleotides can be added to the solution (e.g., an RT master mix) described herein.

In some embodiments, the blocker oligonucleotides can be added to one or more solutions (e.g., a permeabilization buffer and/or an RT buffer). In some embodiments, the blocker oligonucleotides can be added more than once (e.g., once, twice, three times or more) to the solutions or buffers described herein.

In some embodiments, the blocker oligonucleotide is about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more nucleotides in length. In some embodiments, the blocker oligonucleotide has regions that are not complementary to an undesirable nucleic acid, so long as such sequences do not substantially affect specific hybridization of the blocker oligonucleotide to the undesirable nucleic acid. In some embodiments, the blocker oligonucleotides are not contiguous, such that while they may collectively hybridize across a length of the undesirable RNA there may exist gaps between the individual blocker oligonucleotides. For example, in some embodiments, the blocker oligonucleotides that target an undesirable RNA are spaced at least one, at least two, at least 5, at least 10, at least 20, at least 30, at least 50, at least 100, at least 200, at least 300, at least 400, at least 500, at least 600, at least 700, at least 800, at least 900, at least 1000, at least 2000, at least 3000, at least 4000, at least 5000, at least 6000, at least 7000, at least 8000, at least 9000, at least 10000, or more nucleotides apart along the length of the undesirable RNA. As such, there may be a plurality of blocker oligonucleotides that will hybridize adjacent to, or non-contiguous to, each other along the length, or partially along the length, of the undesirable nucleic acid.

In some instances, the blocker oligonucleotides are not associated with, or conjugated to, a detectable label, an optical label, and/or a label as described herein. In some instances, the blocker oligonucleotides are not conjugated to a biotin molecule.

In some embodiments, the blocker oligonucleotide is associated with (e.g., conjugated to) a detectable label, an optical label, and/or a label as described herein. In some instances, the detectable label is a radioisotope, a fluorescent or chemiluminescent moiety, with an enzyme or ligand, which can be used for detection or confirmation that the probe has hybridized to the target sequence. The detectable label can be directly detectable by itself (e.g., radioisotope labels or fluorescent labels) or, in the case of an enzymatic label, can be indirectly detectable, e.g., by catalyzing chemical alterations of a chemical substrate compound or composition, which chemical substrate compound or composition is directly detectable. The detectable label can be qualitatively detected (e.g., optically or spectrally), or it can be quantified using methods known in the art and/or disclosed herein. In some instances, the biological sample is imaged using methods described herein to qualitatively or quantitatively evaluate and detect the blocker oligonucleotides in the sample.

In some embodiments, at least 1, at least 2, at least 3, at least 4, at least 5, at least 6, at least 7, at least 8, at least 9, at least 10, about 15, about 20, about 25, about 30, about 35, about 40, about 45, about 50, about 75, about 100, or more blocker oligonucleotides can hybridize to various sequences, e.g., conserved sequences, of the same undesirable nucleic acid molecule. In some embodiments, the various sequences are identical. In some embodiments, the various sequences are different. In some embodiments, the methods described herein comprise a plurality of blocker oligonucleotides, and the plurality of blocker nucleotides comprise at least 1, at least 2, at least 3, at least 4, at least 5, at least 6, at least 7, at least 8, at least 9, at least 10, about 15, about 20, about 25, about 30, about 35, about 40, about 45, about 50, about 75, about 100, or more blocker oligonucleotides. In some embodiments, the blocker oligonucleotides have the same sequence. In some embodiments, the blocker oligonucleotides have different sequences.

In some embodiments, the plurality of blocker oligonucleotides described herein can target at least 1, at least 2, at least 3, at least 4, at least 5, at least 6, at least 7, at least 8, at least 9, at least 10, at least 20, at least 30, at least 40, at least 50, at least 60, at least 70, at least 80, at least 90, at least 100, at least 200, at least 300, at least 400, at least 500, at least 600, at least 700, at least 800, at least 900, at least 1000, or more undesired nucleic acids. In some embodiments, the blocker oligonucleotide can target at least 1, at least 2, at least 3, at least 4, at least 5, at least 6, at least 7, at least 8, at least 9, at least 10, or more isoforms (or fragments thereof) transcribed from the same gene.

In some embodiments, each blocker oligonucleotide comprises one or more, e.g., at least 1, 2, 3, 4, 5, 6, 7, 8, 9, 10, 11, 12, 13, 14, 15, 16, 17, 18, 19, 20, or more, non-natural nucleic acids. In some embodiments, the blocker oligonucleotide comprises at least 1%, at least 2%, at least 3%, at least 4%, at least 5%, at least 6%, at least 7%, at least 8%, at least 9%, at least 10%, at least 20%, at least 30%, at least 40%, at least 50%, at least 60%, at least 70%, at least 80%, at least 90%, or 100% non-natural nucleic acids. In some embodiments, the blocker oligonucleotide has a melting temperature (Tm) of about 55° C., about 56° C., about 57° C., about 58° C., about 59° C., about 60° C., about 61° C., about 62° C., about 63° C., about 64° C., about 65° C., about 65° C., about 66° C., about 67° C., about 68° C., about 69° C., about 70° C., or higher.

In some embodiments, the non-natural nucleic acid comprises one or more phosphate analogs, for example, phosphodiester analogs (e.g., phosphorothioate, boranophosphate, or phosphonate). In some embodiments, the non-natural nucleic acid comprises one or more aptamers, e.g., substituents at C-2′ (e.g., F, NH2, O-methyl) or modifications to the 5-position of uracil. In some embodiments, the non-natural nucleic acid comprises one or more nucleobase analogs, e.g., 2-thioT, ψT, yT, xA, yC, xC, or xG. In some embodiments, the non-natural nucleic acid comprises substitutions of a furanose ring, e.g., threose nucleic acid (TNA), locked nucleic acid (LNA), or hexitol nucleic acid (HNA). In some embodiments, the non-natural nucleic acid comprises acyclic analogues, e.g., flexible nucleic acid (FNA), glycerol nucleic acid (GNA), or peptide nucleic acid (PNA). Detailed descriptions can be found, e.g., in Appella et al., Current Opinion in Chemical Biology, 13.5-6 (2009): 687-696, which in incorporated herein by reference in its entirety.

In some embodiments, the blocker oligonucleotide includes a single-stranded oligonucleotide having a sequence partially or completely complementary to an undesirable nucleic acid and specifically hybridizes to the undesirable nucleic acid. In some embodiments, the blocker oligonucleotide is at least 70%, at least 75%, at least 80%, at least 85%, at least 90%, at least 91%, at least 92%, at least 93%, at least 94%, at least 95%, at least 96%, at least 97%, at least 98%, or at least 99% complementary to the undesirable nucleic acid. In some embodiments, the blocker oligonucleotide is 100% (i.e., completely) complementary to the undesirable nucleic acid.

The blocker oligonucleotide can be produced by techniques known in the art. For example, in some embodiments, the blocker oligonucleotide can be produced using chemical synthesis, in vitro expression from recombinant nucleic acid molecules, in vivo expression from recombinant nucleic acid molecules, or combinations thereof. The blocker oligonucleotide may also be produced by amplification of the undesirable nucleic acid, e.g., RT-PCR, asymmetric PCR, or rolling circle amplification, followed by incorporation of one or more non-natural nucleic acids.

(i) Locked Nucleic Acid Blocker Oligonucleotides

In some embodiments, a blocker oligonucleotide described herein comprises one or more non-natural nucleic acids. In some embodiments, the non-natural nucleic acid is a locked nucleic acid (LNA). Locked nucleic acids are a type of nucleic acid analog that contains a 2′-O, 4′-C methylene bridge, which increases the affinity for complementary RNA or DNA. In some instances, compared to naturally-occurring oligonucleotides, LNAs provide enhanced stability, increased melting temperature, and binding affinity. This bridge-locked in the 3′-endo conformation restricts the flexibility of the ribofuranose ring and locks the structure into a rigid bicyclic formation. LNAs are used to increase the sensitivity and specificity of molecular biology tools such as DNA microarrays and LNA-based oligonucleotides are being developed as antisense therapies. LNAs can be incorporated into the blocker oligonucleotides by standard phophoramidite chemistry.

In some embodiments, the LNA is a 2′-O,4′-C-methylene-α-1-ribofuranose (α-L-LNA) or a 2′-O,4′-C-methylene-β-d-ribofuranose (β-D-LNA). In some embodiments, the blocker oligonucleotide includes an all-LNA, a LNA mixmer (any combination of LNA and DNA residues), a LNA gapmer (with a central DNA moiety flanked by LNA-modified 5′- or 3′-end), an LNA-modified LNAzyme, or any combinations thereof. Detailed descriptions of LNA can be founds, e.g., in Grünweiler and Roland, BioDrugs 21.4 (2007): 235-243, which is incorporated by reference in its entirety.

In some embodiments, the blocker oligonucleotide comprises one or more (e.g., 1, 2, 3, 4, 5, 6, 7, 8, 9, 10, or more) modifications to its structure. For example, the modifications include one or more carbon moieties attached to the LNA. In some embodiments, the modifications include modified bases, e.g., 2′-O-methoxy-ethyl Bases (2′-MOE) (e.g., 2-MethoxyEthoxy A, 2-MethoxyEthoxy MeC, 2-MethoxyEthoxy G, and/or 2-MethoxyEthoxy T); 2′-O-Methyl RNA Bases (e.g., 2′-O-Methyl RNA Bases); Fluoro Bases (e.g., Fluoro C, Fluoro U, Fluoro A, and/or Fluoro G); 2-Aminopurine, 5-Bromo dU, deoxyUridine, 2,6-Diaminopurine (2-Amino-dA), Dideoxy-C, deoxyInosine, Hydroxymethyl dC, inverted dT, Iso-dG, Iso-dC, inverted Dideoxy-T, 3′-3′-inverted thymine, 5-Methyl dC, 5-Nitroindole, Super T (5-hydroxybutynl-2′-deoxyuridine), Super G (8-aza-7-deazaguanosine) and/or combinations thereof.

In some embodiments, the blocker oligonucleotide comprises a mixture of DNA, RNA and/or LNA bases. In some embodiments, the LNA is about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length. In some instances, the LNA is about 5 to 20, about 10 to 20, or about 15 to 20 nucleotides in length. It is appreciated that the length of an LNA can be adjusted to modulate (e.g., increase) the melting temperature (Tm) so that it is not displaced during other methods disclosed herein (e.g., reverse transcription of the probe and/or analyte).

In some instances, the LNA is complementary to a region of an undesirable RNA molecule. In some instances, the LNA is about 60%, 65%, 70%, 75%, 80%, 85%, 90%, 91%, 92%, 93%, 94%, 95%, 96%, 97%, 98%, 99%, or 100% complementary to a region of an undesirable RNA molecule. Thus, in some instances, the LNA specifically binds (e.g., hybridizes) to a complementary (e.g., 60%, 65%, 70%, 75%, 80%, 85%, 90%, 91%, 92%, 93%, 94%, 95%, 96%, 97%, 98%, 99%, or 100%) to the undesirable RNA molecule.

In some embodiments, each LNA can increase melting temperature (Tm) of the blocker oligonucleotide by at least 1° C., at least 2° C., at least 3° C., at least 4° C., or at least 5° C. as compared to a reference naturally-occurring oligonucleotide with the same sequence. In some embodiments, Tm of the blocker oligonucleotide comprising one or more LNAs is about 10%, about 20%, about 30%, about 40%, about 50%, about 60%, about 70%, about 80%, about 90%, about 100%, or higher than Tm of a reference oligonucleotide with the same sequence except that the LNA is replaced by the corresponding oligonucleotide. In some embodiments, the higher Tm of the blocker oligonucleotide comprising one or more LNAs leads to its increased binding affinity to the undesirable nucleic acid. In some embodiments, the blocker oligonucleotide comprising one or more LNAs has a similar Tm compared to a reference oligonucleotide sharing an identical sequence without LNAs, in which case the blocker oligonucleotide is at least 1, 2, 3, 4, 5, 6, 7, 8, 9, 10, 11, 12, 13, 14, 15, 16, 17, 18, 19, 20 nucleotides shorter than the reference oligonucleotide. In some embodiments, the length of the blocker oligonucleotide, and number and/or positions of incorporated LNAs are determined according to the blocker oligonucleotide's Tm. In some embodiments, the Tm of the blocker oligonucleotide is determined according to the highest temperature used during the spatial analysis workflow (e.g., extension or amplification).

In some embodiments, the one or more LNAs within the blocker oligonucleotide can increase its binding affinity to the undesirable nucleic acid by at least 1 fold, at least 5 fold, at least 10 fold, at least 20 fold, at least 30 fold, at least 40 fold, at least 50 fold, at least 60 fold, at least 70 fold, at least 80 fold, at least 90 fold, at least 100 fold, or more as compared to the binding affinity of the same blocker oligonucleotide except that the LNA is replaced by the corresponding DNA. In some embodiments, because of the increased binding affinity to the undesirable nucleic acid, the blocker oligonucleotide does not disassociate from the undesirable nucleic acid during the extension (e.g., reverse transcription) and/or the amplification (e.g., PCR amplification) steps following the spatial analysis workflow.

In some embodiments, the blocker oligonucleotide includes a degenerate sequence. In some instances, the blocker oligonucleotide described herein comprises one or more (e.g., 1, 2, 3, 4, 5, 6, 7, 8, 9, 10, or more) degenerate sequences. In some embodiments, the degenerate sequence can be a degenerate nucleotide sequence. In some embodiments, the degenerate nucleotide sequence includes about or at least 1, 2, 3, 4, 5, 6, 7, 8, 9, 10, 11, 12, 13, 14, 15, 16, 17, 18, 19, 20, 25, 30, 35, 40, 45, or 50 nucleotides. In some embodiments, the degenerate nucleotides are at least or about 5%, 10%, 20%, 30%, 40%, 50%, 60%, 70%, 80%, 90%, or 100% of the blocker oligonucleotide. In some embodiments, the degenerate sequences are flanked by non-degenerate sequences on one end or on both ends. In some embodiments, the degenerate nucleotides comprise the non-natural nucleic acid (e.g., the LNA) described herein.

(b) Undesirable Nucleic Acid

As used herein, the term “undesirable nucleic acid”, or “undesirable nucleic acid molecule”, refers to an undesired nucleic acid that can specifically or non-specifically bind to the capture probe described herein. In some embodiments, the population of one or more undesirable nucleic acids (e.g., ribosomal RNAs) can be at least 5%, at least 10%, at least 15%, at least 20%, at least 25%, at least 30%, at least 35%, at least 40%, or more of the total nucleic acids (e.g., total RNAs) of the biological sample. In some embodiments, the methods described herein can decrease the population of the one or more undesirable nucleic acids bound to the capture probes to less than 40%, less than 35%, less than 30%, less than 25%, less than 20%, less than 15%, less than 10%, less than 9%, less than 8%, less than 7%, less than 6%, less than 5%, less than 4%, less than 3%, less than 2%, or less than 1% of the total nucleic acids of the biological sample bound to the capture probes. Consequently, the enriched population of the nucleic acid targets of interest bound to the capture probes may comprise at least 99%, 98%, 97%, 96%, 95%, 94%, 93%, 92%, 91%, 90%, 89%, 88%, 87%, 86%, 85%, 84%, 83%, 82%, 81%, or 80%, or any range therein, of the total nucleic acids in the sample that are bound to the capture probes.

In some embodiments, the undesirable nucleic acids (e.g., ribosomal RNAs) can contribute at least 5%, at least 10%, at least 15%, at least 20%, at least 25%, at least 30%, at least 35%, at least 40%, or more of the total number of identified analytes (e.g., sequencing reads) of the biological sample, and the methods described herein can decrease the contribution of the undesirable nucleic acids to less than 40%, less than 35%, less than 30%, less than 25%, less than 20%, less than 15%, less than 10%, less than 9%, less than 8%, less than 7%, less than 6%, less than 5%, less than 4%, less than 3%, less than 2%, or less than 1% of the total number of identified analytes (e.g., sequencing reads) of the biological sample.

In some embodiments, the undesirable nucleic acid is an undesirable RNA molecule. In some embodiments, examples of the undesirable RNA molecule include, but are not limited to, messenger RNA (mRNA), ribosomal RNA (rRNA), mitochondrial RNA (mtRNA), nuclear RNA, chloroplast RNA, transfer RNA (tRNA), microRNA (miRNA), bacterial RNA, and viral RNA. In some embodiments, the undesirable RNA can be a transcript (e.g., present in a tissue section). The undesirable RNA can be small (e.g., less than 200 nucleic acid bases in length) or large (e.g., RNA greater than 200 nucleic acid bases in length).

In some embodiments, the undesirable RNA molecule includes 5.8S ribosomal RNA (rRNA), 5S rRNA, transfer RNA (tRNA), microRNA (miRNA), small nucleolar RNA (snoRNAs), Piwi-interacting RNA (piRNA), tRNA-derived small RNA (tsRNA), and small rDNA-derived RNA (srRNA), or mitochondrial RNA (mtRNA). In some embodiments, the undesirable RNA molecule includes an RNA molecule that is added (e.g., transfected) into a sample (e.g., a small interfering RNA (siRNA)). The undesirable RNA can be double-stranded RNA or single-stranded RNA. In embodiments where the undesirable RNA is double-stranded it is processed as a single-stranded RNA prior to hybridization with the blocker oligonucleotides. In some embodiments, the undesirable RNA can be circular RNA. In some embodiments, the undesirable RNA can be a bacterial rRNA (e.g., 16s rRNA or 23s rRNA). In some embodiments, the undesirable RNA is from E. coli.

In some embodiments, the undesirable RNA molecule is rRNA. In some embodiments, the rRNA is eukaryotic rRNA. In some embodiments, the rRNA is cytoplasmic rRNA. In some embodiments, the rRNA is mitochondrial rRNA. Cytoplasmic rRNAs include, for example, 28S, 26S, 26S, 5.8S, 5S, and 18S rRNAs. Mitochondrial rRNAs include, for example, 12S and 16S rRNAs. The rRNA may also be prokaryotic rRNA, which includes, for example, 5S, 16S, and 23S rRNA. The sequences for rRNAs are well known to those skilled in the art and can be readily found in sequence databases such as GenBank or may be found in the literature. For example, the sequence for the human 18S rRNA can be found in GenBank as Accession No. M10098 and the human 28S rRNA as Accession No. M11167.

In some embodiments, the undesirable RNA molecule is mitochondrial RNA. Mitochondrial RNAs include, for example, 12S rRNA (encoded by MT-RNR1), and 16S rRNA (encoded by MT-RNR2), RNAs encoding electron transport chain proteins (e.g., NADH dehydrogenase, coenzyme Q-cytochrome c reductase/cytochrome b, cytochrome c oxidase, ATP synthase, or humanin), and tRNAs (encoded by MT-TA, MT-TR, MT-TN, MT-TD, MT-TC, MT-TE, MT-TQ, MT-TG, MT-TH, MT-TI, MT-TL1, MT-TL2, MT-TK, MT-TM, MT-TF, MT-TP, MT-TS1, MT-TS2, MT-TT, MT-TW, MT-TY, or MT-TV).

In some embodiments, the undesirable RNA is a plant RNA, e.g., a chloroplast RNA. In some embodiments, the undesirable RNA is from an organism that contains a chloroplast. In some embodiments, the chloroplast RNA can be encoded by the chloroplast genome. For example, the chloroplast RNA can be encoded from genes in chloroplast for rRNAs, tRNAs, ribosomal proteins, RNA polymerase subunits, thylakoid proteins, RuBisCo (Ribulose-1,5-bisphosphate carboxylase/oxygenase) subunits, and proteins mediating redox reactions to recycle electrons. Detailed descriptions can be found, e.g., in Harris et al., Microbiol. Mol. Biol. Rev. 58.4 (1994): 700-754, which is incorporated herein by reference in its entirety.

In some instances, the undesirable RNA is selected from one or more of the group consisting of rRNA, tRNA, snRNA, snoRNA and abundant protein coding mRNA. In some embodiments, the undesirable RNA is transfer RNA (tRNA). In some embodiments, the undesirable RNA may be a particular mRNA. For example, it may be desirable to remove cellular transcripts that are usually present in abundance. Thus, the undesirable mRNA may include, but is not limited to, transcripts encoding hemoglobin (e.g., HBA1/2, HBB, HBD, HBM, HBG1/2, HBE1, HBQ1, and/or HBZ transcripts); Ribulose-1,5-bisphosphate carboxylase/oxygenase (e.g., RuBisCo transcripts); glyceraldehyde-3-phosphate dehydrogenase (e.g., GAPDH transcripts), beta actin (e.g., ACTB transcripts) and/or beta tubulin (e.g., TUBB transcripts). Additional undesirable, abundant protein-coding mRNAs that can be targeted by one or more blocker oligonucleotides include ACTB, B2M, GAPDH, GUSB, HPRT1, HSP90AB1, LDHA, NONO, PGK1, PPIH, RPLP0, TFRC, or any combination thereof.

Other sequences for tRNA and specific mRNA are well known to those skilled in the art and can be readily found in sequence databases such as GenBank or may be found in the literature.

In some embodiments, mRNA is not targeted by the blocker oligonucleotides. In some embodiments, one or more blocker oligonucleotides do not have a poly-dT that will hybridize to the poly-A tail of eukaryotic mRNA. In yet another particular embodiment, the blocker oligonucleotide targets and specifically hybridizes to human 18S or human 28S rRNA. Examples of the sequence of blocker oligonucleotides targeting the full length sequence of human 18S and human 28S rRNA are illustrated in, e.g., US Appl. Publ. No. 2011/0111409 A1, which is incorporated herein by reference.

In some embodiments, the blocker oligonucleotide targets an undesirable nucleic acid pathogen (i.e., non-host) in the biological sample. In some instances, the blocker oligonucleotide targets undesirable non-host RNA from the biological sample. In some embodiments, undesirable non-host nucleic acids targeted by blocker oligonucleotides include one or more of certain bacteria (e.g., Gram-negative bacteria or Gram-positive bacteria), mycobacteria, mycoplasma, fungi, and parasitic cells. In some embodiments, undesirable non-host nucleic acids are indicative of a pathogen, a parasite, a commensal organism, or a symbiont.

In some embodiments, the one or more undesirable RNA molecules are from a single species of RNA. For example, in some embodiments, the one or more undesirable RNA molecules are ribosomal RNA molecules. In some embodiments, the one or more undesirable RNA molecules are mitochondrial RNA molecules. In some embodiments, the undesirable RNA molecules can be a combination of two or more species of RNA. In certain embodiments, the number of different undesirable RNA species to which blocking oligonucleotides are complementary is at least 2, at least 3, at least 4, or at least 5, at least 10, at least 20, at least 30, at least 40, at least 50, at least 75, at least 100, at least 200, at least 300, at least 400, or at least 500, and/or at most 1,000,000, at most 500,000, at most 100,000, at most 50,000, at most 10,000, at most 9000, at most 8000, at most 7000, at most 6000, at most 5000, at most 4000, at most 3000, or at most 2000, such as from 2 to 1,000,000, from 100 to 500,000, from 500 to 100,000, and from 1000 to 10,000. In some embodiments, the undesirable RNA molecule is an RNA fragment of any one of the undesirable RNA molecules described herein. In some embodiments, the undesirable RNA molecule is a full length RNA molecule of any one of the undesirable RNA molecules described herein.

In some embodiments, the blocker oligonucleotide comprises a sequence that is substantially complementary to a conserved sequence in the undesirable nucleic acid. For example, the conserved sequence is substantially similar among species, including for example one or more of humans, dogs, cats, cows, chickens, rabbits, mice, rats, sheep, horses, amphibians, and reptiles.

In some embodiments, the blocker oligonucleotides are designed to target tissue-specific undesirable RNA molecules. In some instances, and without limitation, the blocker oligonucleotides are designed to target undesirable RNA molecules expressed in one or more of skeletal muscle tissue, heart tissue, liver tissue, pancreas tissue, brain tissue, lung tissue, kidney tissue, breast tissue, skin tissue, uterus tissue, ovary tissue, bladder tissue, bone tissue, stomach tissue, esophagus tissue, colon tissue, or any combination thereof. In some instances, the blocker oligonucleotides are designed to target blood-specific undesirable RNA molecules. In some instances, the blocker oligonucleotides target transcripts encoding one of more hemoglobin genes in blood.

In some embodiments, the blocker oligonucleotides are designed to target species-specific undesirable RNA molecules (e.g., human RNAs or plant RNAs). In some instances, the blocker oligonucleotides target human-specific undesirable RNA molecules. In some instances, the blocker oligonucleotides target mammal-specific undesirable RNA molecules. In some instances, the blocker oligonucleotides target animal-specific undesirable RNA molecules. In some instances, the blocker oligonucleotides target plant-specific undesirable RNA molecules. Examples of mammals include, without limitation, a non-primate (e.g., cow, pig, horse, cat, dog, rat, etc.) or a primate (e.g., monkey or human). In some embodiments, the blocker oligonucleotides are designed to target one or more species-specific undesirable RNA molecules from a pet or farm animal.

In some embodiments, the blocker oligonucleotides are designed to target organelle-specific undesirable RNA molecules (e.g., chloroplast RNAs, ribosomal RNAs, or mitochondrial RNAs). In some embodiments, the blocker oligonucleotides are designed to target function-specific undesirable RNA molecules (e.g., RNAs involved in protein synthesis).

In some embodiments, the blocker oligonucleotides can be designed to target one or more (e.g., 1, 2, 3, 4, 5, or more) conserved or homologous regions within the undesirable RNA molecule. For example, identification of the conserved or homologous regions can be conducted by sequence alignment using various algorithms known in the art. In some embodiments, the blocker oligonucleotides can be designed to target one or more (e.g., 1, 2, 3, 4, 5, or more) shared regions among multiple isoforms of the undesirable RNA molecule. In some embodiments, the blocker oligonucleotides can be designed to target one or more (e.g., 1, 2, 3, 4, 5, or more) adenosine-rich regions of the undesirable RNA molecule.

In some embodiments, the undesirable nucleic acid binds (e.g., hybridizes) to the capture probe specifically. In some embodiments, the undesirable nucleic acid binds to the capture probe non-specifically. In some embodiments, the undesirable nucleic acid binds to a surface of the spatial array (e.g., a slide) via non-specific interactions (e.g., because of high viscosity of rRNA). In some embodiments, the blocker oligonucleotide can bind to the undesirable nucleic acid to reduce its specific, or non-specific binding to the capture probe.

In some embodiments, the blocker oligonucleotide can competitively bind (e.g., hybridize) to and release the undesirable nucleic acid that has already bound to the capture probe. For example, a blocker oligonucleotide can comprise a sequence that is complementary to an adenosine-rich region within the undesirable nucleic acid. When the binding affinity between the blocker oligonucleotide and the undesirable nucleic acid is higher than the binding affinity between the undesirable nucleic acid and the capture probe, the undesirable nucleic acid can disassociate from the capture probe and then hybridize to the blocker oligonucleotide.

In some embodiments, the blocker oligonucleotide hybridizes to a first region (e.g., a conserved region) within the undesirable nucleic acid, and the capture probe hybridizes to a second region (e.g., an adenosine-rich region) within the undesirable nucleic acid. In such cases, the first region can stop the undesirable nucleic acid from being extended, e.g., by reverse transcription. In addition, the first region can stop the undesirable nucleic acid from being amplified, e.g., by PCR amplification.

In some embodiments, hybridization of the blocker oligonucleotide to the undesirable nucleic acid (e.g., rRNA) can disrupt the structure (e.g., tertiary structure) of the undesirable nucleic acid (e.g., due to conformational change), such that the blocker oligonucleotide-hybridized undesirable nucleic acid does not bind to the capture probe, or disassociate from the capture probe upon hybridization with the blocker oligonucleotide. For example, the capture probe-binding region within the undesirable nucleic acid becomes inaccessible upon hybridization with the blocker oligonucleotide.

In some embodiments, once the blocker oligonucleotide binds (e.g., hybridizes) to the undesirable nucleic acid, the blocker oligonucleotide does not disassociate from the undesirable nucleic acid during the extension (e.g., reverse transcription) and/or the amplification (e.g., PCR amplification) steps following the spatial analysis workflow. As a result, the undesirable nucleic acid cannot be extended and/or amplified. In some embodiments, the blocker oligonucleotide has a Tm that is higher than the highest temperature used during the spatial analysis workflow (e.g., extension or amplification), such that the blocker oligonucleotide does not disassociate from the undesirable nucleic acid once bound. In some embodiments, the biological sample is an FFPE sample and the Tm of the blocker oligonucleotide is compatible with the de-crosslinking step (e.g., the Tm is higher than the temperature used for de-cross linking).

In some embodiments, the blocker oligonucleotide-undesirable nucleic acid complex is removed by one or more washing steps following the spatial analysis workflow. For example, the washing step can occur after the extension step, in order to remove unincorporated nucleotides which can interfere with subsequent steps.

(c) Hybridization of the Blocker Oligonucleotide to the Undesirable Nucleic Acid

In some embodiments, one or more blocker oligonucleotides hybridize to an undesirable nucleic acid. In some embodiments, one or more blocker oligonucleotides hybridize to one or more portions of a sequence of the undesirable nucleic acid. In some embodiments, one or more blocker oligonucleotides hybridize to the complete sequence of the undesirable nucleic acid. Hybridization can occur at an undesirable nucleic acid having a sequence that is 100% complementary to the blocker oligonucleotide(s). In some embodiments, hybridization can occur at a sequence in the undesirable nucleic acid that is at least (e.g., at least about) 80%, at least (e.g., at least about) 85%, at least (e.g., at least about) 90%, at least (e.g., at least about) 95%, at least (e.g., at least about) 96%, at least (e.g., at least about) 97%, at least (e.g., at least about) 98%, or at least (e.g., at least about) 99% complementary to the blocker oligonucleotide(s).

In some embodiments, the blocker oligonucleotide may be complementary to all or part of an undesirable RNA sequence and therefore, there may be more than one blocker oligonucleotide that specifically hybridizes to the undesirable nucleic acid. For example, there may be at least 1, 2, 3, 4, 5, 6, 7, 8, 9, 10, or more blocker oligonucleotides that specifically hybridize to an undesirable nucleic acid. In some embodiments, the undesirable nucleic acid has a tertiary structure and the blocker oligonucleotide can be complementary to an exposed portion of the undesirable nucleic acid sequence.

In some embodiments, one or more blocker oligonucleotides can hybridize to the undesirable nucleic acid such that less than 1%, at least about 1%, 2%, 3%, 4%, 5%, 6%, 7%, 8%, 9%, 10%, at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, or 100% of the complete sequence of the undesirable nucleic acid is hybridized by the blocker oligonucleotides.

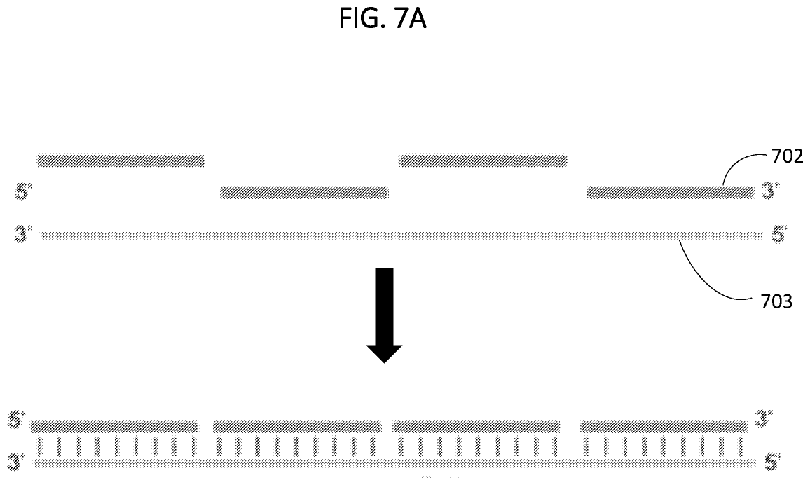

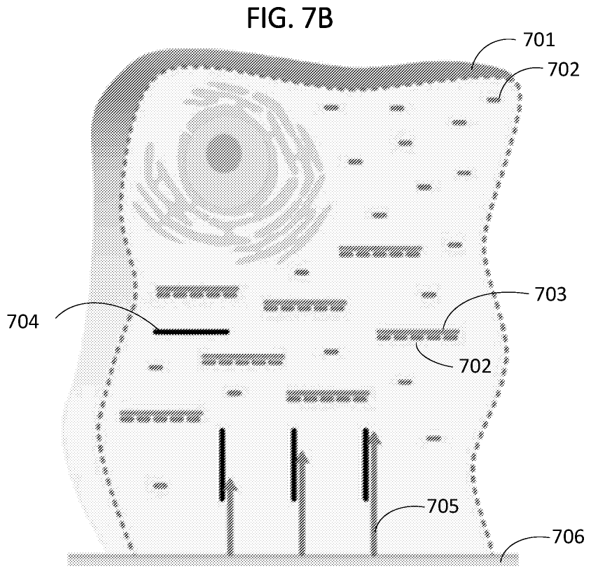

Referring to FIG. 7A, in some instances, a blocker oligonucleotide 702 is designed to be complementary to a section of an undesirable target 703. In some instances, as shown in FIG. 7A, multiple blocker oligonucleotides 702 can be designed to target the same undesirable target 703. In some instances, the blocker oligonucleotide specifically binds (e.g., hybridizes) to an undesirable target 703. In some embodiments, the blocker oligonucleotide 702 comprises at least one non-natural nucleic acid (e.g., one or more LNAs).

In some embodiments, the blocker oligonucleotides may be complementary to sequences that overlap one another. In some embodiments, the blocker oligonucleotides may be complementary to non-overlapping sequences.

(d) Methods of Blocking Undesirable Nucleic Acid Binding to Capture Probes

In some embodiments, provided herein are methods for identifying a location of an analyte of interest (e.g., any of the analyte described herein) in a biological sample (e.g., any of the biological samples described herein) comprising (a) contacting the biological sample with a substrate (e.g., any of the substrate described herein) comprising a plurality of capture probes, wherein a capture probe (e.g., any of the capture probe described herein) of the plurality of capture probes comprises (i) a spatial barcode (e.g., any of the spatial barcode described herein) and (ii) a capture domain (e.g., any of the capture domain described herein) that specifically hybridizes to the analyte of interest; (b) contacting the biological sample with a plurality of blocker oligonucleotides, wherein a blocker oligonucleotide (e.g., any of the blocker oligonucleotide described herein) of the plurality of blocker oligonucleotides is substantially complementary to a sequence of an undesirable nucleic acid molecule (e.g., any of the undesirable nucleic acid molecule described herein) in the biological sample; (c) hybridizing the blocker oligonucleotide to the undesirable nucleic acid molecule; (d) extending (e.g., using any of the extending methods described herein) the capture probe using the analyte of interest as a template to generate an extended capture probe; and (e) amplifying (e.g., using any of the amplifying methods described herein) the extended capture probe.

In some embodiments, provided herein are methods for increasing the efficiency of capture of an analyte of interest (e.g., any of the analyte described herein) in a biological sample (e.g., any of the biological samples described herein) comprising (a) contacting the biological sample with a substrate (e.g., any of the substrate described herein) comprising a plurality of capture probes, wherein a capture probe (e.g., any of the capture probe described herein) of the plurality of capture probes comprises (i) a spatial barcode (e.g., any of the spatial barcode described herein) and (ii) a capture domain (e.g., any of the capture domain described herein) that specifically hybridizes to the analyte of interest; (b) contacting the biological sample with a plurality of blocker oligonucleotides, wherein a blocker oligonucleotide (e.g., any of the blocker oligonucleotide described herein) of the plurality of blocker oligonucleotides is substantially complementary to a sequence of an undesirable nucleic acid molecule (e.g., any of the undesirable nucleic acid molecule described herein) in the biological sample; (c) hybridizing the blocker oligonucleotide to the undesirable nucleic acid molecule; (d) extending (e.g., using any of the extending methods described herein) the capture probe using the analyte of interest as a template to generate an extended capture probe; and (e) amplifying (e.g., using any of the amplifying methods described herein) the extended capture probe. In some embodiments, the efficiency of capture of the analyte of interest is increased relative to a reference biological sample to which the plurality of blocker oligonucleotides has not been added.

While not intending to be bound by any theory, it is believed that one skilled in the art would appreciate that the order of the steps described above can be changed according to specific experimental needs.

Referring to FIG. 7B, the methods include a biological sample 701 (e.g., a single cell, a tissue sample, etc.). In some instances, the biological sample is placed on a substrate 706 that includes a plurality of probes 705. In some instances, the sample is permeabilized using one or more of the permeabilization buffers or solutions disclosed herein. In some instances, a plurality of blocker oligonucleotides 702 are added to the biological sample. The plurality of blocker oligonucleotides 702 hybridize to an undesirable nucleic acid molecule (e.g., rRNA) 703 but does not hybridize to an analyte of interest 704. After blocker oligonucleotides 702 hybridize to the undesirable nucleic acid molecule 703, analytes of interest (e.g., 704) are captured by a probe 705 on the substrate 706 for analysis of the analyte (e.g., as disclosed herein).

In some instances, the blocker oligonucleotides are added to the biological sample at substantially the same time as the substrate is contacted to the sample. In some instances, the blocker oligonucleotides are added to the sample prior to the time when the substrate is contacted to the sample. In some instances, the biological sample is permeabilized before the blocker oligonucleotides are added to the sample and/or before the biological sample is contacted with the substrate.

In some instances, the blocker oligonucleotides are added to the biological sample before the permeabilization step. In some instances, blocker oligonucleotides are added to the biological sample during steps that involve staining of the biological sample. In some instances, the blocker oligonucleotides are added to the biological sample at substantially the same time as the permeabilization step. In some instances, the blocker oligonucleotides are added to the biological sample after the permeabilization step.

In some instances, blocker oligonucleotides are added at multiple steps during analyte capture. For example, blocker oligonucleotides can be added to the biological sample before adding a permeabilization buffer, at the same time when a permeabilization buffer is added, after a permeabilization buffer is added, during hybridization of analytes of interest to the capture probes, or after hybridization of the capture probes.

In some instances, blocker oligonucleotides are included in the same solution as the permeabilization buffer. In some instances, blocker oligonucleotides are included in the same solution as an RT buffer (e.g., an RT mastermix buffer).

In some embodiments, the undesirable nucleic acids (e.g., any of the undesirable nucleic acids described herein) are blocked (e.g., hybridized) by the plurality of blocker oligonucleotides before one or more target analytes (e.g., target nucleic acids or analytes of interest) are hybridized to the capture probes. In some embodiments, the undesirable nucleic acids (e.g., any of the undesirable nucleic acids described herein) are blocked (e.g., hybridized) by the plurality of blocker oligonucleotides at substantially the same time as one or more target analytes (e.g., target nucleic acids or analytes of interest) are hybridized to the capture probes. In some embodiments, the undesirable nucleic acids (e.g., any of the undesirable nucleic acids described herein) are blocked (e.g., hybridized) by the plurality of blocker oligonucleotides after one or more target analytes (e.g., target nucleic acids or analytes of interest) are hybridized to the capture probes.

In some embodiments, the analyte of interest includes a capture probe binding domain (e.g., a poly(A) sequence), which can hybridize to a capture probe (e.g., a capture probe immobilized, directly or indirectly, on a substrate). In some embodiments, methods provided herein include contacting a biological sample with a substrate, where the capture probe is affixed to the substrate (e.g., immobilized to the substrate, directly or indirectly). In some embodiments, the capture probe includes a spatial barcode and the capture domain. In some embodiments, the capture probe binding domain of the analyte of interest specifically binds to the capture domain. After hybridization of the analyte of interest to the capture probe, the analyte of interest is extended at the 3′ end to make a copy of the additional components (e.g., the spatial barcode) of the capture probe. In some embodiments, methods of analyte of interest capture as provided herein include permeabilization of the biological sample such that the capture probe and/or a blocker oligonucleotide can more easily hybridize to the captured analyte of interest (i.e., compared to no permeabilization). In some embodiments, reverse transcription (RT) reagents and/or blocker oligonucleotides are added to permeabilized biological samples. Incubation with the RT reagents can produce spatially-barcoded full-length cDNA from the captured analytes (e.g., polyadenylated mRNA). Second strand reagents (e.g., second strand primers, enzymes) can be added to the biological sample on the slide to initiate second strand synthesis.

The resulting cDNA can be denatured from the capture probe template and transferred (e.g., to a clean tube) for amplification, and/or library construction as described herein. The spatially-barcoded, full-length cDNA can be amplified via PCR prior to library construction. The cDNA can then be enzymatically fragmented and size-selected in order to optimize the cDNA amplicon size. P5, P7, i7, and i5 can be incorporated into the library as for downstream sequencing, and additional library sequencing regions, such as TruSeq Read 2, can be added via End Repair, A-tailing, Adaptor Ligation, and PCR. The cDNA fragments can then be sequenced using paired-end sequencing using TruSeq Read 1 and TruSeq Read 2 as sequencing primer sites. In some instances, the cDNA library is sequenced using any method described herein, such that different sequencing domains specific to other sequencing methods and techniques can be incorporated into a capture probe or introduced during library preparation. In some instances, the sequence of the extended probe and/or analyte is determined via sequencing. In some instances, the spatial barcode is sequenced, providing the location of the analyte.

In some embodiments, the methods described herein can provide multiple benefits (e.g., less expensive, and/or less time-consuming) as compared to other methods (e.g., depleting the undesirable nucleic acid by beads comprising complementary sequences of the undesirable nucleic acid) for increasing efficiency of identifying a location of an analyte (e.g., an analyte of interest) in a biological sample. In some embodiments, results (e.g., sequencing reads) using the methods described herein are more consistent (e.g., less variations) as compared to the other methods (e.g., depleting the undesirable nucleic acid by beads comprising complementary sequences of the undesirable nucleic acid) for increasing efficiency of identifying a location of an analyte (e.g., an analyte of interest) in a biological sample. For example, the methods described herein (e.g., adding blocker oligonucleotides to the RT master mix) comprise less steps as compared to the other methods (e.g., depleting the undesirable nucleic acid by beads comprising complementary sequences of the undesirable nucleic acid), such that more target nucleic acids can be saved without adhering to additional surfaces (e.g., the beads described herein).

(e) Kits

In some embodiments, also provided herein are kits that include one or more reagents to detect one or more analytes of interest described herein. In some instances, the kit includes a substrate comprising a plurality of capture probes comprising a spatial barcode and the capture domain. In some instances, the kit includes a plurality of blocker nucleotides described herein.

A non-limiting example of a kit used to perform any of the methods described herein includes: a) an array comprising a plurality of capture probes; b) a plurality of blocker oligonucleotides, wherein a blocker oligonucleotide of the plurality of blocker oligonucleotides is substantially complementary to a sequence of an undesirable nucleic acid molecule in a biological sample, wherein the blocker oligonucleotide comprises at least one non-natural nucleic acid; c) one or more permeabilization agents; and d) instructions for using the kit.

### Examples

The invention is further described in the following examples, which do not limit the scope of the invention described in the claims.

### Example 1. Blocking Ribosomal RNA Using Blocker Oligonucleotides Comprising Locked Nucleic Acids

As a non-limiting example as shown in FIG. 7A, a blocker oligonucleotide 702 can be designed to be complementary to a section (e.g., a conserved region) of a ribosomal RNA (rRNA) (indicated as 703 herein). Multiple blocker oligonucleotides 702 can be designed to target different regions of the same rRNA 703. The blocker oligonucleotide can comprise one or more locked nucleic acids (LNAs) described here, and can specifically hybridize to the rRNA 703. As shown in FIG. 7B, a cell sample (indicated as 701 herein) can be placed on a substrate 706 that includes a plurality of probes 705. The sample can be permeabilized using one or more of the permeabilization buffers or solutions disclosed herein. After permeabilization or concurrently with permeabilization, a plurality of blocker oligonucleotides 702 can be added to the biological sample. The plurality of blocker oligonucleotides 702 can hybridize to the rRNA (or other undesirable RNA or DNA species) 703 but does not hybridize to an analyte of interest 704. As the blocker oligonucleotides 702/rRNA 703 hybridize, the analytes of interest (e.g., 704) can be captured by a probe 705 on the substrate 706 for analysis of the analyte (e.g., as disclosed herein). Because of its high melting temperature (Tm), the blocker oligonucleotide 702 does not disassociate from the rRNA 703 throughout the analysis of the analyte, e.g., during extension (e.g., by reverse transcription) of the probe 705 or amplification (e.g., by PCR amplification) of the extended probe using the methods disclosed herein. In addition, the blocker oligonucleotide 702/rRNA 703 hybrid can be washed away during the analysis of the analyte. Thus, the blocker oligonucleotide 702 can prevent the rRNA 703 from binding to the probe 705, which in turn can increase sequencing reads of the analyte of interest 704.

### Example 2. Blocking Mitochondrial RNA Using Blocker Oligonucleotides Comprising Locked Nucleic Acids

As a non-limiting example as shown in FIG. 7A, a blocker oligonucleotide 702 can be designed to be complementary to a section (e.g., a conserved region) of a mitochondrial RNA (mtRNA) (indicated as 703 herein). Multiple blocker oligonucleotides 702 can be designed to target different regions of the same mtRNA 703. The blocker oligonucleotide can comprise one or more locked nucleic acids (LNAs) and can specifically hybridize to the mtRNA 703. As shown in FIG. 7B, a cell sample (indicated as 701 herein) can be placed on a substrate 706 that includes a plurality of probes 705. The sample can be permeabilized using one or more of the permeabilization buffers or solutions disclosed herein. After permeabilization or concurrently with permeabilization, a plurality of blocker oligonucleotides 702 can be added to the biological sample. The plurality of blocker oligonucleotides 702 can hybridize to the mtRNA 703 but does not hybridize to an analyte of interest 704. As the blocker oligonucleotides 702/mtRNA 703 hybridize, the analytes of interest (e.g., 704) can be captured by a probe 705 on the substrate 706 for analysis of the analyte (e.g., as disclosed herein). Because of its high melting temperature (Tm), the blocker oligonucleotide 702 does not disassociate from the mtRNA 703 throughout the analysis of the analyte, e.g., during extension (e.g., by reverse transcription) of the probe 705 or amplification (e.g., by PCR amplification) of the extended probe using the methods disclosed herein. In addition, the blocker oligonucleotide 702/mtRNA 703 can be washed away during the analysis of the analyte. Thus, the blocker oligonucleotide 702 can prevent the mtRNA 703 from binding to the probe 705, which in turn can increase sequencing reads of the analyte of interest 704.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1.-64. (canceled)

65. A method for increasing the efficiency of capture of an analyte of interest in a biological sample comprising:
(a) contacting the biological sample with a substrate comprising a plurality of capture probes, wherein a capture probe of the plurality of capture probes comprises (i) a spatial barcode and (ii) a capture domain that specifically hybridizes to the analyte of interest;
(b) contacting the biological sample with a plurality of blocker oligonucleotides, wherein a blocker oligonucleotide of the plurality of blocker oligonucleotides comprises (i) at least one non-natural nucleic acid and (ii) is substantially complementary to a sequence of an undesirable nucleic acid molecule in the biological sample;
(c) hybridizing the blocker oligonucleotide to the undesirable nucleic acid molecule and the analyte of interest to the capture domain of the capture probe on the array;
(d) extending the capture probe using the analyte of interest as a template to generate an extended capture probe; and
(e) amplifying the extended capture probe,
wherein the efficiency of capture of an analyte of interest is increased relative to a reference biological sample to which the plurality of blocker oligonucleotides has not been added.

66. The method of claim 65, further comprising determining (i) the sequence of the spatial barcode or a complement thereof, and (ii) all or a portion of the sequence of the analyte of interest from the biological sample, and using the sequences of (i) and (ii) to identify the location of the analyte of interest in the biological sample.

67. The method of claim 65, wherein the non-natural nucleic acid is a locked nucleic acid.

68. The method of claim 65, wherein the blocker oligonucleotide comprises one or more modifications to its structure.

69. The method of claim 65, wherein the blocker oligonucleotide comprises one or more degenerate nucleotides.

70. The method of claim 65, wherein the blocker oligonucleotide comprises a sequence that is complementary to a conserved sequence in the undesirable nucleic acid molecule.

71. The method of claim 65, wherein the blocker oligonucleotide comprises at least 1% non-natural nucleic acids.

72. The method of claim 65, wherein the plurality of blocker oligonucleotides comprises blocker oligonucleotides that target at least 2 undesired nucleic acid.

73. The method of claim 65, wherein the blocker oligonucleotide is about 25 to about 50 nucleotides in length.

74. The method of claim 65, wherein the blocker oligonucleotide has a melting temperature of about 55° C. to about 70° C.

75. The method of claim 65, wherein the blocker oligonucleotide is substantially complementary to all or a portion of the sequence of the undesirable nucleic acid molecule in the biological sample.

76. The method of claim 65, wherein the undesirable nucleic acid molecule is an RNA molecule.

77. The method of claim 76, wherein the RNA molecule is a transfer RNA, a ribosomal RNA, a mitochondrial RNA, nuclear RNA, chloroplast RNA, cytoplasmic RNA, or any combinations thereof.

78. The method of claim 65, wherein the undesirable nucleic acid molecule is one or more of an mRNA that encodes hemoglobin, ribulose-1,5-bisphosphate carboxylase/oxygenase, or glyceraldehyde-3-phosphate dehydrogenase.

79. The method of claim 65, further comprising removing the hybridization products of the plurality of blocker oligonucleotide and undesirable nucleic acid molecules.

80. The method of claim 65, wherein the biological sample is permeabilized prior to or concurrently with contacting the biological sample with the plurality of blocker oligonucleotides, wherein the biological sample is permeabilized with pepsin or proteinase K.

81. The method of claim 65, wherein the biological sample is a tissue sample.

82. The method of claim 65, wherein the analyte of interest is mRNA.

83. A kit comprising:
(a) an array comprising a plurality of capture probes;
(b) a plurality of blocker oligonucleotides, wherein a blocker oligonucleotide of the plurality of blocker oligonucleotides comprises (i) at least one non-natural nucleotide and (ii) is substantially complementary to a sequence of an undesirable nucleic acid molecule in a biological sample;
(c) one or more permeabilization agents; and
(d) instructions for performing the method of claim 65.

84. A system for decreasing the capture of an undesirable nucleic acid molecule from a biological sample, comprising:
(a) an array comprises a plurality of capture probes, wherein a capture probe of the plurality of capture probes comprises (i) a spatial barcode and (ii) a capture domain that specifically hybridizes to an analyte of interest from the biological sample; and
(b) a plurality of blocker oligonucleotides, wherein a blocker oligonucleotide of the plurality of blocker oligonucleotides comprises (i) at least one non-natural nucleic acid and (ii) is substantially complementary to a sequence of the undesirable nucleic acid molecule from the biological sample.

85. The system of claim 84, further comprising a first substrate and a second substrate, wherein the biological sample is mounted on the first substrate, and the array is mounted on the second substrate.

86. The system of claim 84, wherein the first substrate is aligned with the second substrate, such that at least a portion of the biological sample is aligned with at least a portion of the array.
